(19) United States
(12) Patent Application Publication
(10) Pub. No.: US 2018/0071394 A1
(43) Pub. Date: Mar. 15, 2018

(54) POLYESTER POLYMER MATRICES FOR THE DELIVERY OF ALLERGENS

(71) Applicant: Selecta Biosciences, Inc., Watertown, MA (US)

(72) Inventors: Conlin O'Neil, Andover, MA (US); Petr Ilyinskii, Cambridge, MA (US)

(21) Appl. No.: 15/685,648

(22) Filed: Aug. 24, 2017

Related U.S. Application Data
(60) Provisional application No. 62/379,745, filed on Aug. 26, 2016.
(60) Provisional application No. 62/379,742, filed on Aug. 25, 2016.

(51) Int. Cl. A61K 47/34; A61K 39/35; A61K 9/107

(52) U.S. Cl. CPC A61K 47/34; A61K 9/107; A61K 39/35

(57) ABSTRACT

Provided herein are polyester polymer synthetic nanocarriers that encapsulate allergens as well as methods of their use and production.

### RELATED APPLICATIONS

This application claims the benefit of priority under 35 U.S.C. § 119 of U.S. Provisional Application Nos. 62/379,742 filed Aug. 25, 2016, and 62/379,745, filed Aug. 26, 2016, the entire contents of each of which are incorporated herein by reference.

### FIELD OF THE INVENTION

Provided herein are polyester polymer synthetic nanocarriers that encapsulate allergens as well as methods of their use and production.

### SUMMARY OF THE INVENTION

In one aspect, compositions comprising synthetic nanocarriers comprising a polyester polymer matrix and allergen, wherein the allergen is encapsulated in the polyester polymer matrix, and wherein the polyester polymer matrix has a calculated hydrophilic to lipophilic balance (HLB) ranging from 11 to 15 are provided. In one embodiment of any one of the compositions provided, the load of the allergen is 0.5 to 2.5 wt %. In one embodiment of any one of the compositions provided, the compositions further comprise a pharmaceutically acceptable excipient.

In one embodiment of any one of the compositions provided, the HLB is from 11 to 13. In one embodiment of any one of the compositions provided, the HLB is from 11 to 12. In one embodiment of any one of the compositions provided, the HLB is from 11.5 to 14. In one embodiment of any one of the compositions provided, the HLB is from 11.5 to 13. In one embodiment of any one of the compositions provided, the HLB is from 11.5 to 12. In one embodiment of any one of the compositions provided, the HLB is 12 to 14.5. In one embodiment of any one of the compositions provided, the HLB is 12 to 14. In one embodiment of any one of the compositions provided, the HLB is 12 to 13.5. In one embodiment of any one of the compositions provided, the HLB is 12 to 13. In one embodiment of any one of the compositions provided, the HLB is 12.5 to 14.5. In one embodiment of any one of the compositions provided, the HLB is 12.5 to 14. In one embodiment of any one of the compositions provided, the HLB is 12.5 to 13.5. In one embodiment of any one of the compositions provided, the HLB is 12.5 to 13. In one embodiment of any one of the compositions provided, the HLB is 11. In one embodiment of any one of the compositions provided, the HLB is 12. In one embodiment of any one of the compositions provided, the HLB is 13. In one embodiment of any one of the compositions provided, the HLB is 14. In one embodiment of any one of the compositions provided, the HLB is 15.

In one embodiment of any one of the compositions provided, the allergen is a plant allergen, insect allergen, insect sting allergen, animal allergen, latex allergen, mold allergen, fungal allergen, cosmetic allergen, drug allergen, food allergen, or dust allergen. In one embodiment of any one of the compositions provided, the allergen is a pollen, ragweed, bee sting, wasp sting, hornet sting, yellow jacket sting, house dust mite, cockroach, pet, milk, egg, nut, fish, shellfish, soy, legume, seed, or wheat allergen. In one embodiment of any one of the compositions provided, the allergen is in the form of a purified protein. In one embodiment of any one of the compositions provided, the allergen is in the form of a mixture of purified proteins. In one embodiment of any one of the compositions provided, the allergen is in the form of an extract. In one embodiment of any one of the compositions provided, the extract is a peanut extract, wheat protein extract, ragweed extract, egg extract or dust mite extract.

In one embodiment of any one of the compositions provided, the weighted mean retention time of a sample of the allergen, obtained using reverse-phase high performance liquid chromatography (RP-HPLC), is between 1 and 10 minutes. In one embodiment of any one of the compositions provided, the weighted mean retention time of a sample of the allergen is between 2 and 10 minutes, between 3 and 10 minutes, between 4 and 10 minutes, between 5 and 10 minutes, between 6 and 10 minutes, between 7 and 10 minutes, between 8 and 10 minutes or between 9 and 10 minutes. In one embodiment of any one of the compositions provided, the weighted mean retention time of a sample of the allergen is between 2 and 9 minutes, between 3 and 9 minutes, between 4 and 9 minutes, between 5 and 9 minutes, between 6 and 9 minutes, between 7 and 9 minutes or between 8 and 9 minutes. In one embodiment of any one of the compositions provided, the weighted mean retention time of a sample of the allergen is between 2 and 8 minutes, between 3 and 8 minutes, between 4 and 8 minutes, between 5 and 8 minutes, between 6 and 8 minutes or between 7 and 8 minutes. In one embodiment of any one of the compositions provided, the weighted mean retention time of a sample of the allergen is between 2 and 7 minutes, between 3 and 7 minutes, between 4 and 7 minutes, between 5 and 7 minutes or between 6 and 7 minutes. In one embodiment of any one of the compositions provided, the weighted mean retention time of a sample of the allergen is between 2 and 6 minutes, between 3 and 6 minutes, between 4 and 6 minutes or between 5 and 6 minutes. In one embodiment of any one of the compositions provided, the weighted mean retention time of a sample of the allergen is between 2 and 5 minutes, between 3 and 5 minutes or between 4 and 5 minutes. In one embodiment of any one of the compositions provided, the weighted mean retention time of a sample of the allergen is between 2 and 4 minutes or between 3 and 4 minutes. In one embodiment of any one of the compositions provided, the weighted mean retention time of a sample of the allergen is between 2 and 3 minutes. In one embodiment of any one of the compositions provided, the weighted mean retention time is as calculated according to Equation 3.

In one embodiment of any one of the compositions provided, the RP-HPLC is performed on an ultra high pressure liquid chromatography instrument (UHPLC). In one embodiment of any one of the compositions provided, the UHPLC instrument is an Agilent UHPLC instrument.

In one embodiment of any one of the compositions provided, the sample of the allergen is monitored at 200 nm absorbance using RP-HPLC.

In one embodiment of any one of the compositions provided, the sample of the allergen injected onto a column of the RP-HPLC instrument contains 3 μg of allergen. In one embodiment of any one of the compositions provided, the column is a C18 UHPLC column. In one embodiment of any one of the compositions provided, the column is an XBridge Peptide BEH C18 UHPLC column.

In one embodiment of any one of the compositions provided, the flow rate of the RP-HPLC is 3.0 mL/minute.

In one embodiment of any one of the compositions provided, the mobile phase A of the RP-HPLC is composed of 94.9% water, 5% acetonitrile, and 0.1% trifluoroacetic acid on a volume percent basis. In one embodiment of any one of the compositions provided, the mobile phase B of the RP-HPLC is composed of 19.9% water, 80% acetonitrile, 0.1% trifluoroacetic acid on a volume percent basis.

In one embodiment of any one of the compositions provided, the water for the RP-HPLC is supplied by a reverse osmosis deionized water system and 0.2 μm filtered.

In one embodiment of any one of the compositions provided, the protein peaks are identified, and the area under the curve is calculated using chromatography software. In one embodiment of any one of the compositions provided, the weight percent of each peak is calculated based on the area under the curve and is divided by the sum of the area under the peaks for all identified protein peaks. In one embodiment of any one of the compositions provided, the chromatography software is Agilent chromatography software.

In one embodiment of any one of the compositions provided, the load of the allergen is 0.5 to 2 wt %. In one embodiment of any one of the compositions provided, the load of the allergen is 0.5 to 1.5 wt %. In one embodiment of any one of the compositions provided, the load of the allergen is 0.5 to 1 wt %. In one embodiment of any one of the compositions provided, the load of the allergen is 1 to 2.5 wt %. In one embodiment of any one of the compositions provided, the load of the allergen is 1 to 2 wt %. In one embodiment of any one of the compositions provided, the load of the allergen is 1 to 1.5 wt %. In one embodiment of any one of the compositions provided, the load of the allergen is 1.5 to 2.5 wt %. In one embodiment of any one of the compositions provided, the load of the allergen is 1.5 to 2 wt %. In one embodiment of any one of the compositions provided, the load of the allergen is 0.5 wt %. In one embodiment of any one of the compositions provided, the load of the allergen is 1 wt %. In one embodiment of any one of the compositions provided, the load of the allergen is 1.5 wt %. In one embodiment of any one of the compositions provided, the load of the allergen is 2 wt %. In one embodiment of any one of the compositions provided, the load of the allergen is 2.5 wt %.

In one embodiment of any one of the compositions provided, the polyester polymer matrix comprises poly(lactic acid) (PLA), poly(glycolic acid) (PGA), poly(lactic-co-glycolic acid) (PLGA), polyvalerolactone (PVL), or polycaprolactone (PCL). In one embodiment of any one of the compositions provided, the polyester polymer matrix further comprises a block copolymer. In one embodiment of any one of the compositions provided, the block copolymer comprises a polyester coupled to a polyether. In one embodiment of any one of the compositions provided, the block copolymer or polyether comprises polyethylene glycol. In one embodiment of any one of the compositions provided, when the block copolymer comprises PLGA and PEG, the wt % of the PEG is no more than 5 wt %. In one embodiment of any one of the compositions provided, when the block copolymer comprises PLGA and PEG, the wt % of the PEG is no more than 4 wt %. In one embodiment of any one of the compositions provided, when the block copolymer comprises PLGA and PEG, the wt % of PEG is no more than 3.75 wt %. In one embodiment of any one of the compositions provided, when the block copolymer comprises PLA and PEG, the wt % of the PEG is from 0 to 15 wt %. In one embodiment of any one of the compositions provided, when the block copolymer comprises PLA and PEG, the wt % of the PEG is 13.5 wt %. In one embodiment of any one of the compositions provided, when the block copolymer comprises PLA and PEG, the wt % of the PEG is from 0 to 14.5 wt %, 0 to 14 wt %, 0 to 13.5 wt % or 1 to 13.5 wt %.

In one embodiment of any one of the compositions provided, the mean dimension of the synthetic nanocarriers in the composition, obtained using dynamic light scattering, is greater than 90 nm but less than 200 nm. In one embodiment of any one of the compositions provided, the mean dimension is greater than 100 nm but less than 200 nm. In one embodiment of any one of the compositions provided, the mean dimension is greater than 110 nm but less than 200 nm. In one embodiment of any one of the compositions provided, the mean dimension is greater than 110 nm but less than 170 nm. In one embodiment of any one of the compositions provided, the mean dimension is greater than 120 nm but less than 200 nm. In one embodiment of any one of the compositions provided, the mean dimension is greater than 150 nm but less than 200 nm. In one embodiment of any one of the compositions provided, the mean dimension is greater than 90 nm but less than 175 nm. In one embodiment of any one of the compositions provided, the mean dimension is greater than 100 nm but less than 175 nm. In one embodiment of any one of the compositions provided, the mean dimension is greater than 110 nm but less than 175 nm. In one embodiment of any one of the compositions provided, the mean dimension is greater than 120 nm but less than 175 nm. In one embodiment of any one of the compositions provided, the mean dimension is greater than 150 nm but less than 175 nm.

In one embodiment of any one of the compositions provided, the compositions further comprise a Th1-biasing adjuvant. In one embodiment of any one of the compositions provided, the adjuvant is comprised in different synthetic nanocarriers. In one embodiment of any one of the compositions provided, the adjuvant is comprised in the same synthetic nanocarriers.

In one embodiment of any one of the compositions provided, the synthetic nanocarriers are double emulsion synthetic nanocarriers.

In one aspect, a method is provided comprising providing any one of the compositions provided herein to a subject.

In one aspect, a method is provided comprising administering any one of the compositions provided herein to a subject.

In one aspect, a method comprising producing any one of the compositions comprising synthetic nanocarriers provided herein is provided. In one embodiment of any one of the methods provided, the method further comprises providing the composition to a subject. In one embodiment of any one of the methods provided, the method further comprises administering the composition to a subject.

In one embodiment of any one of the methods provided, the producing comprises calculating the HLB of the synthetic nanocarriers. In one embodiment of any one of the methods provided, the HLB is calculated according to any one of the methods provided herein. In one embodiment of any one of the methods provided, the producing comprises determining the weighted mean retention time of a sample of the allergen. In one embodiment of any one of the methods provided herein, the weighted mean retention time is determined according to any one of the methods provided herein. In one embodiment of any one of the methods provided herein, the weighted mean retention time of a sample of the allergen is determined using RP-HPLC, such as according to any one of the methods provided herein. In one embodiment of any one of the methods provided, the producing comprises steps of a double emulsion method.

In one aspect, a method comprising selecting any one of the compositions provided herein, and providing the composition to a subject is provided. In one aspect, a method comprising selecting any one of the compositions provided herein, and administering the composition to a subject is provided.

In one embodiment of any one of the methods provided, the method further comprises producing synthetic nanocarriers. In one embodiment of any one of the methods provided, the producing comprises calculating the HLB of the synthetic nanocarriers. In one embodiment of any one of the methods provided, the HLB is calculated according to any one of the methods provided herein. In one embodiment of any one of the methods provided, the producing comprises determining the weighted mean retention time of a sample of the allergen. In one embodiment of any one of the methods provided herein, the weighted mean retention time is determined according to any one of the methods provided herein. In one embodiment of any one of the methods provided herein, the weighted mean retention time of a sample of the allergen is determined using RP-HPLC, such as according to any one of the methods provided herein. In one embodiment of any one of the methods provided, the producing comprises steps of a double emulsion method.

In one aspect, a composition comprising synthetic nanocarriers produced by any one of the methods provided herein is provided.

In one aspect, a method comprising providing any one of the compositions produced by any one of the methods provided herein to a subject is provided.

In one aspect, a method comprising administering any one of the compositions produced by any one of the methods provided herein to a subject is provided.

In one embodiment of any one of the methods provided, the subject is in need thereof.

In one aspect, a use of any one of the compositions provided herein for the manufacture of a medicament is provided. In one embodiment, the medicament is for use as provided in any one of the methods provided herein. In one embodiment, the medicament is for providing the composition to a subject. In one embodiment, the medicament is for administering the composition to a subject. In one embodiment of any one of the uses provided, the subject is in need thereof.

In one aspect, a composition as provided in any one of the Examples is provided.

In one aspect, a method as provided in any one of the Examples is provided.

### BRIEF DESCRIPTION OF THE FIGURES

FIG. 1 is a schematic depicting an allergen-containing nanocarrier.

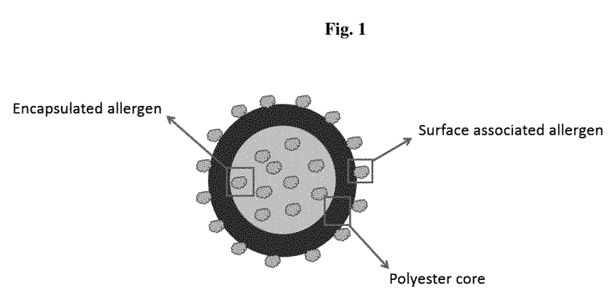

FIG. 2 is a chart showing a correlation between the concentration of raw wheat (RW) gliadin allergen (as measured by the quantities (mg) of RW gliadin allergen added to the formulation divided by the formed nanocarrier surface area) and the surface presentation of the allergen on the nanocarriers (as measured by surface ELISA values).

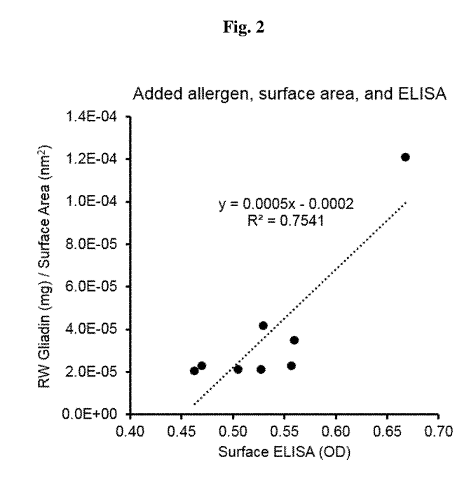

FIGS. 3A-3B include charts showing a linear relationship between the concentration of allergen (as measured by quantities of allergen added to the formulation divided by the formed nanocarrier surface area) and the surface presentation of the allergen (as determined by surface ELISA values). FIG. 3A includes a chart showing a correlation between the concentration of House Dust Mite (HDM) and HDM surface presentation. FIG. 3B includes a chart showing a correlation between the concentration of Ragweed and Ragweed surface presentation.

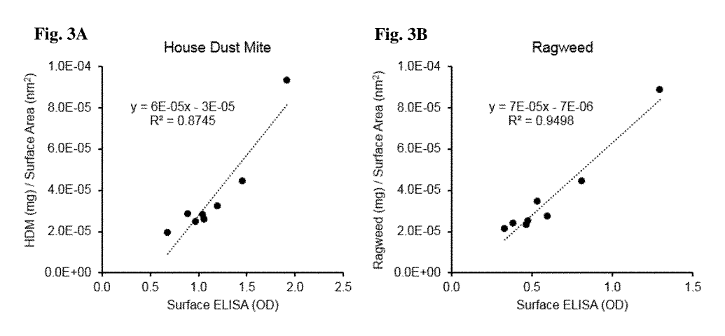

FIG. 4 provides an example of a UV/vis adsorption spectrum for a protein. Source: elte.prompt.hu/sites/default/files/tananyagok/IntroductionToPracticalBiochemistry/ch04s06.html.

FIG. 5 provides an example UV/vis absorption spectrum of a RP-HPLC chromatogram peak from an ovalbumin (OVA) allergen solution injection (6.518 minutes retention time).

### DETAILED DESCRIPTION OF THE INVENTION

Before describing the present invention in detail, it is to be understood that this invention is not limited to particularly exemplified materials or process parameters as such may, of course, vary. It is also to be understood that the terminology used herein is for the purpose of describing particular embodiments of the invention only, and is not intended to be limiting of the use of alternative terminology to describe the present invention.

All publications, patents and patent applications cited herein, whether supra or infra, are hereby incorporated by reference in their entirety for all purposes.

As used in this specification and the appended claims, the singular forms “a,” “an” and “the” include plural referents unless the content clearly dictates otherwise. For example, reference to “a polymer” includes a mixture of two or more such molecules or a mixture of differing molecular weights of a single polymer species, reference to “a synthetic nanocarrier” includes a mixture of two or more such synthetic nanocarriers or a plurality of such synthetic nanocarriers, and the like.

As used herein, the term “comprise” or variations thereof such as “comprises” or “comprising” are to be read to indicate the inclusion of any recited integer (e.g. a feature, element, characteristic, property, method/process step or limitation) or group of integers (e.g. features, elements, characteristics, properties, method/process steps or limitations) but not the exclusion of any other integer or group of integers. Thus, as used herein, the term “comprising” is inclusive and does not exclude additional, unrecited integers or method/process steps.

In embodiments of any one of the compositions and methods provided herein, “comprising” may be replaced with “consisting essentially of” or “consisting of”. The phrase “consisting essentially of” is used herein to require the specified integer(s) or steps as well as those which do not materially affect the character or function of the claimed invention. As used herein, the term “consisting” is used to indicate the presence of the recited integer (e.g. a feature, element, characteristic, property, method/process step or limitation) or group of integers (e.g. features, elements, characteristics, properties, method/process steps or limitations) alone.

### A. Introduction

It has surprisingly been found that there is an optimal hydrophilic to lipophilic balance (HLB) for synthetic nanocarriers made up of a polyester polymer matrix and comprising allergen, a material that can be somewhat hydrophobic. Generally, without wishing to be bound by a particular theory, when the HLB is too hydrophobic, the load of allergen that is comprised within the synthetic nanocarriers is low to negligible, as the allergen is lost during the formulation of the synthetic nanocarriers. On the other hand, again without wishing to be bound by a particular theory, when the HLB is too hydrophilic, more hydrophobic materials, will be lost due to a lack of an impermeable membrane or surface with the right properties for strong adsorption. It has been found that an HLB in an optimal range can result in synthetic nanocarriers with a desired load of allergen with less of the allergen being displayed on the surface of the synthetic nanocarriers. This is significant at least because the potential for serious reactions to allergen when delivered with synthetic nanocarriers can be minimized as recognition of or exposure to the allergen molecules until they reach their site of action can be reduced.

In addition, the physiochemical properties of the allergens may also play a role in the production of the synthetic nanocarriers as provided herein and/or the in vivo effects of use of such synthetic nanocarriers. Without wishing to be bound to any particular theory, allergens can possess certain physiochemical properties that protect their structure from the effects of temperature, pH, surfactants, and/or proteolytic enzymes. These can be accomplished via a number of structural strategies such as stabilization by disulfide bonds, glycosylation, glycation, structural aggregation, and the appearance of repetitive structures. Further, many allergens can bind to lipids and membranes to enable transportation and protection from destructive physiological environments.

Thus, provided herein are synthetic nanocarrier compositions that are optimized. Synthetic nanocarriers have been developed that encapsulate allergens at a substantial load and efficiency while minimizing the amount of allergen present on the nanocarrier surface. These synthetic nanocarriers comprise a polyester polymer matrix and have a hydrophilic to lipophilic balance that is in an appropriate range to accomplish the aforementioned optimization. In some embodiments, the allergens exhibit a weighted mean retention time within a specific range when measured by RP-HPLC. The present invention is directed to such synthetic nanocarrier compositions as well as methods of their production and use.

### B. Definitions

“Adjuvant” mean an agent that stimulates an immune response to an antigen, such as an allergen, but is not the antigen or derived from the antigen. “Stimulate”, as used herein, refers to inducing, enhancing, directing, or redirecting an immune response. Such agents include agents that boost an immune response to an antigen but is not the antigen or derived from the antigen. Preferably, the adjuvants herein, in some embodiments, are Th1-biasing adjuvants.

“Th1-biasing adjuvant” means an agent that (1) biases an immune response from a response that is characterized by a Th2-type cytokine response to a response that is characterized by a Th1-type cytokine response, or (2) amplifies a suboptimal and/or ineffective Th1-type response. In certain embodiments, Th1-biasing adjuvants may be interleukins, interferon, cytokines, etc. In specific embodiments, a Th1-biasing adjuvant may be a natural or synthetic agonist for a Toll-like receptor (TLR) such as TLR-1, TLR-2, TLR-3, TLR-4, TLR-5, TLR-6, TLR-7, TLR-8, TLR-9, TLR-10, and TLR-11 agonists. In specific embodiments, synthetic nanocarriers incorporate agonists for toll-like receptors (TLRs) 7 & 8 (“TLR 7/8 agonists”). Of utility are the TLR 7/8 agonist compounds disclosed in U.S. Pat. No. 6,696,076 to Tomai et al., including but not limited to imidazoquinoline amines, imidazopyridine amines, 6,7-fused cycloalkylimidazopyridine amines, and 1,2-bridged imidazoquinoline amines. Preferred Th1-biasing adjuvants comprise imiquimod and R848, in some embodiments. In some embodiments, the R848 is conjugated to a polymer or unit thereof. In other embodiments, the Th1-biasing adjuvant is any one those provided by U.S. Publication No. 2011/0027217; such adjuvants being incorporated herein by reference. Other preferred Th1-biasing adjuvants comprise CpG-containing immunostimulatory nucleic acids, in some embodiments.

“Administering” or “administration” or “administer” means providing a material to a subject in a manner that is pharmacologically useful. The term is intended to include causing to be administered in some embodiments. “Causing to be administered” means causing, urging, encouraging, aiding, inducing or directing, directly or indirectly, another party to administer the material. In some embodiments, the administering is done directly. In other embodiments, the administering is done indirectly whereby based on advice, a prescription or direction provided by a clinician or other medical professional. In such instances, the subject or another individual may administer the composition to his/herself directly.

“Allergen” means a substance that can cause an allergic reaction. Such reactions can be characterized by binding to allergen-specific IgE, activation of IgE receptor bearing cells resulting in a Th2-type pattern of cytokine response, activation of mast cells and/or histamine release, etc. Conditions in which a subject may suffer from such an allergic reaction, are referred to herein as allergies.

Allergens include, but are not limited to, plant allergens (e.g., pollen, ragweed allergen), insect allergens, insect sting allergens (e.g., bee sting allergens), animal allergens (e.g., pet allergens, such as animal dander or cat Fel d 1 antigen), latex allergens, mold allergens, fungal allergens, cosmetic allergens, drug allergens, food allergens, dust, insect venom, viruses, bacteria, etc. Food allergens include, but are not limited to, milk allergens, egg allergens, nut allergens (e.g., peanut or tree nut allergens, etc. (e.g., walnuts, cashews, etc.)), fish allergens, shellfish allergens, soy allergens, legume allergens, seed allergens and wheat allergens. Insect sting allergens include, but are not limited to, allergens that are or are associated with bee stings, wasp stings, hornet stings, yellow jacket stings, etc. Insect allergens include, but are not limited to, house dust mite allergens (e.g., Der P1 antigen) and cockroach allergens. Drug allergens include, but are not limited to, allergens that are or are associated with antibiotics, NSAIDs, anaesthetics, etc. Pollen allergens include, but are not limited to, grass allergens, tree allergens, weed allergens, flower allergens, etc.

Subjects that develop or are at risk of developing an allergic reaction to any one or more of the allergens provided herein may be treated with any one of the compositions and methods provided herein. Subjects that may be treated with any one of the compositions and methods provided also include those who have or are at risk of having an allergy to any one or more of the allergens provided. “Allergens associated with an allergy” are allergens that result in, or would be expected by a clinician to result in, alone or in combination with other allergens, an allergic reaction or a symptom of an allergic response or reaction in a subject. “Type(s) of allergens” means molecules that share the same, or substantially the same, antigenic characteristics in the context of an allergic reaction. In some embodiments of any one of the compositions or methods provided herein, the allergens may be proteins, polypeptides, peptides, or lipoproteins. An allergen can be provided herein in the same form as to what a subject is exposed that causes an allergic reaction but may also be a fragment or derivative thereof. When provided herein in the same form as to what a subject is exposed, the allergen may be a purified protein or mixture of purified proteins or an extract or portion thereof.

An “allergy”, also referred to herein as an “allergic condition,” is a condition where there occurs an undesired immune response, such as a hypersensitivity reaction, to a substance, generally a foreign substance, that does not occur in at least some others in a population. Such substances are referred to herein as allergens. Allergies or allergic conditions include, but are not limited to, allergic asthma, hay fever, hives, eczema, plant allergies, bee sting allergies, pet allergies, latex allergies, mold allergies, cosmetic allergies, food allergies, allergic rhinitis or coryza, topic allergic reactions, anaphylaxis, atopic dermatitis, and other allergic conditions. The allergic reaction may be the result of a hypersensitivity reaction to any allergen. In some embodiments, the allergy is a food allergy. Food allergies include, but are not limited to, milk allergies, egg allergies, nut allergies, fish allergies, shellfish allergies, soy allergies or wheat allergies. Any one of the subjects provided herein may be a subject that has or is at risk of any one of the allergies provided herein.

“Amount effective” in the context of a composition or dose of a composition for administration to a subject refers to an amount of the composition or dose that produces one or more desired responses in the subject, for example, the reduction or amelioration of allergic reactions, such as hypersensitivity reactions. Therefore, in some embodiments, an amount effective is any amount of a composition or dose provided herein that produces one or more of the desired therapeutic effects, including a reduction or elimination of a hypersensitivity reaction, as provided herein. This amount can be for in vitro or in vivo purposes. For in vivo purposes, the amount can be one that a clinician would believe may have a clinical benefit for a subject in need thereof. Any one of the compositions as provided herein can be in an amount effective.

Amounts effective can involve reducing the level of an undesired immune response, although in some embodiments, it involves preventing an undesired immune response altogether. Amounts effective can also involve delaying the occurrence of an undesired immune response. An amount that is effective can also be an amount that produces a desired therapeutic endpoint or a desired therapeutic result. In other embodiments, the amounts effective can involve enhancing the level of a desired response, such as a therapeutic endpoint or result. The achievement of any of the foregoing can be monitored by routine methods.

Amounts effective will depend, of course, on the particular subject being treated; the severity of a condition, disease or disorder; the individual patient parameters including age, physical condition, size and weight; the duration of the treatment; the nature of concurrent therapy (if any); the specific route of administration and like factors within the knowledge and expertise of the health practitioner. These factors are well known to those of ordinary skill in the art and can be addressed with no more than routine experimentation. It is generally preferred that a maximum dose be used, that is, the highest safe dose according to sound medical judgment. It will be understood by those of ordinary skill in the art, however, that a patient may insist upon a lower dose or tolerable dose for medical reasons, psychological reasons or for virtually any other reason. In general, doses of the components in the compositions of the invention refer to the amount of the components.

“Average”, as used herein, refers to the arithmetic mean unless otherwise noted.

“Different synthetic nanocarriers” refers to a different population of synthetic nanocarriers distinct from another population of synthetic nanocarriers. For instance, when an adjuvant is comprised in different synthetic nanocarriers from the synthetic nanocarriers that encapsulate allergen, the adjuvant is comprised in a different population of synthetic nanocarriers distinct from the synthetic nanocarriers that encapsulate the allergen. The synthetic nanocarriers of the different population of synthetic nanocarriers may be different in all respects from the other population of synthetic nanocarriers, meaning their structure and component(s) are different. In other embodiments, however, the structure of the synthetic nanocarriers may be the same and all that is different is that the synthetic nanocarriers comprise one or more or all different components, such as the allergen, adjuvant, etc. In still other embodiments, the structure of a different population of synthetic nanocarriers may be different and the component(s) are the same as the other population of synthetic nanocarriers.

“Double emulsion synthetic nanocarriers” refers to synthetic nanocarriers that are made using a double emulsion process. Such processes are known to those in the art and are provided herein. Generally, a double emulsion process includes the production of a water-oil-water emulsion in order to create the synthetic nanocarriers. Any one of the compositions of synthetic nanocarriers provided herein may be made by a double emulsion process. Such synthetic nanocarriers are double emulsion synthetic nanocarriers.

“Encapsulate” means to enclose at least a portion of a substance within a synthetic nanocarrier. In some embodiments, a substance is enclosed completely within a synthetic nanocarrier. In other embodiments, most or all of a substance that is encapsulated is not exposed to the local environment external to the synthetic nanocarrier. In other embodiments, no more than 50%, 40%, 30%, 20%, 10% or 5% (weight/weight) is exposed to the local environment. Encapsulation is distinct from absorption, which places most or all of a substance on a surface of a synthetic nanocarrier, and leaves the substance exposed to the local environment external to the synthetic nanocarrier.

“Extract” refers to a composition obtained from a naturally-available raw material that is somehow processed or purified. In some embodiments, the extract comprises purified allergens(s) in addition to other substances present in the naturally-available raw material. In other embodiments, the extract comprises only the purified allergen(s). However, when the extract comprise only the purified allergen(s), these allergens have not been synthesized but have been purified to such an extent from naturally-available raw material. The extracts provided herein comprise one or more allergens. The extract may be a portion of the naturally-available raw material and may contain a mixture of allergenic and nonallergenic substances. The allergen encapulated within the synthetic nanocarriers provided may be from or in the form of an extract.

Useful extracts, as provided herein, include, but are not limited to, tree (Acacia, Alder, Ash, Bayberry, Beech, Birch, Box Elder, Cedar, Cottonwood, Cypress, Elm, Eucalyptus, Hackberry, Hazelnut, Hickory, Juniper, Maple, Melaleuca, Mesquite, Mulberry, Oak, Olive, Palm, Pecan, Pepper Tree, Pine, Poplar, Privet, Sweetgum, Sycamore, Walnut, etc.), weed (Baccharis, Carelessweed, Cocklebur, Dock, Dog Fennnel, Goldenrod, Kochia, Lambs Quarters, Marshelder, Mugwort, Nettle, Pigweed, Plantain, Ragweed, Russian Thistle, Sagebrush, Saltbush, Sheep Sorrel, Waterhemp, etc.), grass (Bahia, Bermuda, Brome, Johnson, June, Meadow Fescue, Orchard, Quack, Redtop, Rye, Sweet Vernal, Timothy, etc.), pollen (Acacia, Alder, Alfalfa, Ash, Baccharis, Bahia, Bayberry, Beech, Bermuda, Birch, Box Elder, Brome, Carelessweed, Cedar, Cocklebur, Corn, Cottonwood, Cypress, Dock, Dog Fennel, Elm, Eucalyptus, Goldenrod, Hackberry, Hazelnut, Hickory, Johnson, Juniper, June, Kochia, Lambs Quarters, Maple, Marshelder, Meadow Fescue, Melaleuca, Mesquite, Mugwort, Mulberry, Nettle, Oak, Olive, Orchard, Palm, Pecan, Pepper Tree, Pigweed, Pine, Plantain, Poplar, Privet, Quack, Ragweed, Redtop, Russian Thistle, Rye, Sagebrush, Saltbush, Sheep Sorrel, Sweetgum, Sycamore, Timothy, Walnut, Waterhemp, Wheat, Willow, etc.), crop (Alfalfa, Corn, Wheat, etc.), venom (Honey Bee, Yellow Jacket, Yellow Hornet, White-faced Hornet, Wasp, Mixed Vespid, etc.), mold and/or fungi (Acremonium strictum, Alternaria alternata, Aspergillus fumigatus, Aspergillus niger, Aureobasidium pullulans, Botrytis cinerea, Candida albicans, Chaetomium globosum, Cladosporium ciadosporioides, Cladosporium sphaerospermum, Curvularia inaequalis, Drechslera sorokiniana, Epicoccum nigrum, Fusarium roseum, Mucor plumbeus, Penicillin notatum, Phoma herbarum, Rhizopus oryzae, Rhodotorula mucilaginosa, Stemphylium sarcinaeforme, Trichophyton mentagrophytes, Yeast, etc.), smut (Corn, etc.), environmental (Dust Mite, Mite, Cat Hair, Cattle, Dog, Feather, Guinea Pig, Horse, Mouse, Rabbit, Ant, Cockroach, Mosquito, Alfalfa Pollen, Corn Pollen, Cottonseed, Gum, etc.) and food extracts (Almond, Apple, Apricot, Asparagus, Avocado, Banana, Barley, Bean, Beef, Brazil Nut, Broccoli, Buckwheat, Cabbage, Cantaloupe, Carrot, Casein, Celery, Cherry, Chicken, Cinnamon, Clam, Cocoa, Coconut, Codfish, Coffee, Corn, Crab, Cucumber, Egg, Fish Mix, Flounder, Garlic, Grapes, Grapefruit, Halibut, Honeydew, Lamb, Lemon, Lettuce, Lima Bean, Lobster, Milk, Mushroom, Mustard Seed, Oat Grain, Olive, Onion, Orange, Oyster, Pea, Peach, Peanut, Pear, Pecan, Pepper, Pineapple, Pistachio Nut, Plum, Pork, Potato, Rice, Rye, Salmon, Sesame Seed, Mixed Shellfish, Shrimp, Soybean, Spinach, Squash, Strawberry, Tea, Tomato, Tuna, Turkey, Vanilla, Walnut, Watermelon, Wheat, Yeast, etc.). In some embodiments, the extract may be combined with one or more other extracts and the combination or a portion thereof is used for incorporation within the synthetic nanocarriers. Other extracts include, but are not limited to, peanut extract, wheat protein extract, ragweed extract, egg extract or dust mite extract.

Wheat protein extracts include, but are not limited to, those that comprise prolamins or glutelins. Prolamins include, but are not limited to, gliadin. Glutelins include, but are not limited to, glutenin. Wheat protein extracts also include, but are not limited to, those that comprise gluten.

“Hydrophilic to lipophilic balance” or “HLB” refers to the hydrophilic-lipophilic balance of a composition. As used herein, the HLB refers to the hydrophilic-lipophilic balance of the polyester polymer matrix that forms the structure of the synthetic nanocarriers. The HLB may be calculated using Griffin's method or Davie's method or any one of the methods provided herein, such as in the Examples. The HLB value is on a scale from 0 to 20, with 0 corresponding to a completely hydrophobic (lipophilic) composition, and 20 corresponding to a completely hydrophilic (lipophobic) composition. In one embodiment of any one of the compositions or methods provided herein, the HLB value of the composition is as according to Griffin's method. In one embodiment of any one of the compositions or methods provided herein, the HLB value of the composition is as according to Davie's method. In one embodiment of any one of the compositions or methods provided herein, the HLB value is calculated according to any one of the equations or examples as provided herein.

In one embodiment of any one of the compositions or methods provided herein, the HLB is determined using Griffin's method (i.e. by determining the ratio of the hydrophilic molecular weight fraction versus the total molecular weight and multiplying by 20) (Equation 1). The molecular weights may be derived from literature or other known or expected molecular weights, or the molecular weights may be determined by measurement, such as by proton nuclear magnetic resonance spectroscopy (H-NMR). In embodiments where the molecular weights are determined with H-NMR, the Mn and structure of the polymers that were used in the matrix can be obtained. For a polymer mixture, the HLB is a combined HLB determined by multiplying the contribution of each individual polymer by its weight percent in the mixture (Equation 2).

“Load”, as used herein, is the amount of a component relative to the total dry recipe weight of all components of a synthetic nanocarrier (weight/weight). Generally, such a load is calculated as an average across a population of synthetic nanocarriers. In embodiments of any one of the compositions provided herein, the load is calculated as may be described in the Examples or as otherwise known in the art.

“Maximum dimension of a synthetic nanocarrier” means the largest dimension of a nanocarrier measured along any axis of the synthetic nanocarrier. “Minimum dimension of a synthetic nanocarrier” means the smallest dimension of a synthetic nanocarrier measured along any axis of the synthetic nanocarrier. For example, for a spheroidal synthetic nanocarrier, the maximum and minimum dimension of a synthetic nanocarrier would be substantially identical, and would be the size of its diameter. Similarly, for a cuboidal synthetic nanocarrier, the minimum dimension of a synthetic nanocarrier would be the smallest of its height, width, length or diagonal, while the maximum dimension of a synthetic nanocarrier would be the largest of its height, width, length or diagonal. In an embodiment, a minimum dimension of at least 75%, preferably at least 80%, more preferably at least 90%, of the synthetic nanocarriers in a sample, based on the total number of synthetic nanocarriers in the sample, is equal to or greater than 100 nm. In an embodiment, a maximum dimension of at least 75%, preferably at least 80%, more preferably at least 90%, of the synthetic nanocarriers in a sample, based on the total number of synthetic nanocarriers in the sample, is equal to or less than 5 μm. Preferably, a minimum dimension of at least 75%, preferably at least 80%, more preferably at least 90%, of the synthetic nanocarriers in a sample, based on the total number of synthetic nanocarriers in the sample, is greater than 110 nm, more preferably greater than 120 nm, more preferably greater than 130 nm, and more preferably still greater than 150 nm. Aspects ratios of the maximum and minimum dimensions of synthetic nanocarriers may vary depending on the embodiment. For instance, aspect ratios of the maximum to minimum dimensions of the synthetic nanocarriers may vary from 1:1 to 1,000,000:1, preferably from 1:1 to 100,000:1, more preferably from 1:1 to 10,000:1, more preferably from 1:1 to 1000:1, still more preferably from 1:1 to 100:1, and yet more preferably from 1:1 to 10:1.

Preferably, a maximum dimension of at least 75%, preferably at least 80%, more preferably at least 90%, of the synthetic nanocarriers in a sample, based on the total number of synthetic nanocarriers in the sample is equal to or less than 3 μm, more preferably equal to or less than 2 μm, more preferably equal to or less than 1 μm, more preferably equal to or less than 800 nm, more preferably equal to or less than 600 nm, more preferably still equal to or less than 500 nm, more preferably still equal to or less than 300 nm, more preferably still equal to or less than 250 nm and even more preferably still equal to or less than 200 nm. In preferred embodiments, a minimum dimension of at least 75%, preferably at least 80%, more preferably at least 90%, of the synthetic nanocarriers in a sample, based on the total number of synthetic nanocarriers in the sample, is equal to or greater than 90 nm, more preferably equal to or greater than 100 nm, more preferably equal to or greater than 110 nm, more preferably equal to or greater than 120 nm, more preferably equal to or greater than 130 nm, more preferably equal to or greater than 140 nm, and more preferably still equal to or greater than 150 nm. Measurement of synthetic nanocarrier dimensions (e.g., effective diameter) may be obtained, in some embodiments, by suspending the synthetic nanocarriers in a liquid (usually aqueous) media and using dynamic light scattering (DLS) (e.g., using a Brookhaven ZetaPALS instrument). In any one of the compositions or methods provided, the dimensions of the synthetic nanocarriers are so obtained.

For example, a suspension of synthetic nanocarriers can be diluted from an aqueous buffer into purified water to achieve a final synthetic nanocarrier suspension concentration of approximately 0.01 to 0.5 mg/mL. The diluted suspension may be prepared directly inside, or transferred to, a suitable cuvette for DLS analysis. The cuvette may then be placed in the DLS, allowed to equilibrate to the controlled temperature, and then scanned for sufficient time to acquire a stable and reproducible distribution based on appropriate inputs for viscosity of the medium and refractive indicies of the sample. The effective diameter, or mean of the distribution, is then reported. Determining the effective sizes of high aspect ratio, or non-spheroidal, synthetic nanocarriers may require augmentative techniques, such as electron microscopy, to obtain more accurate measurements. “Dimension” or “size” or “diameter” of synthetic nanocarriers means the mean of a particle size distribution, for example, obtained using dynamic light scattering.

“Pharmaceutically acceptable excipient” or “pharmaceutically acceptable carrier” means a pharmacologically inactive material that can be used together with a pharmacologically active material to formulate the compositions. Pharmaceutically acceptable excipients comprise a variety of materials known in the art, including but not limited to saccharides (such as glucose, lactose, and the like), preservatives such as antimicrobial agents, reconstitution aids, colorants, saline (such as phosphate buffered saline), and buffers. Other examples, without limitation, of pharmaceutically acceptable excipients include calcium carbonate, calcium phosphate, various diluents, various sugars and types of starch, cellulose derivatives, gelatin, vegetable oils and polyethylene glycols.

“Polyester polymer matrix” refers to the polymers that make up, at least in part, the structure of the synthetic nanocarriers and at least some of which associate to form a matrix. In some embodiments of any one of the compositions provided herein, such polymers make up the complete structure of the synthetic nanocarriers (not including the components that are delivered using the synthetic nanocarriers, such as the allergen and/or adjuvant). The polymers in the matrix are of one or more types of polymers, at least one of which must be a polyester. As used herein, a “polyester” is a polymer that comprises repeating ester bonds. Polyester polymers include, but are not limited to, PLA, PLGA, PLG, polyvalerolactone, and polycaprolactone. They may also be functionalized or derivatized polyesters. The polyester polymer matrix may include one or more other polymers or units thereof, such as a block copolymer. These other polymers may be or include polyester polymers. “Block copolymer” refers to two or more homopolymer subunits linked by covalent bonds. The union of the homopolymer subunits may require an intermediate non-repeating subunit, known as a junction block. Block copolymers with two or three distinct blocks are called diblock copolymers and triblock copolymers, respectively. In some embodiments, the block copolymer of the polyester polymer matrix is a copolymer of a polyester and a hydrophilic polymer, such as polyethylene glycol (PEG), a peptide polymer or polyacrylic acid. Examples of such copolymers include, but are not limited to, PLA-PEG, PLGA-PEG or PCL-PEG. Preferably, the polyester polymer matrix material suitable for the compositions described herein is selected based on it having an appropriate HLB level. In any one of the methods provided herein, a step of selecting the polyester polymer matrix based on its HLB level may be included. In any one of the methods provided herein, a step of calculating the HLB of a polyester polymer matrix may be included.

“Producing” refers to any action that results in a material being made or being made available. An act of producing includes preparing the material or processing it in some manner. In some embodiments, an act of producing includes any act that makes that material available for use by another. This term is intended to include “causing to produce”. “Causing to produce” means causing, urging, encouraging, aiding, inducing or directing or acting in coordination with an entity for the entity to make a material(s), or make it available, as provided herein. In some embodiments of any one of the methods provided herein, the method may comprise or further comprise any one of the steps of producing as described herein.

“Providing” means an action or set of actions that an individual performs that supplies a material for practicing the invention. Providing may include acts of producing, distributing, selling, giving, making available, prescribing or administering the material. The action or set of actions may be taken either directly oneself or indirectly. Thus, this term is intended to include “causing to provide”. “Causing to provide” means causing, urging, encouraging, aiding, inducing or directing or acting in coordination with an entity for the entity to supply a material for practicing of the present invention. In some embodiments of any one of the methods provided herein, the method may comprise or further comprise any one of the steps of providing as described herein.

“Purified protein” means that the protein is free of other substances to an extent practical and appropriate for its intended use. Purified proteins, generally, are proteins that are sufficiently free from other constituents, such as of a naturally-available raw material from which it can be obtained or derived, so as to be useful in, for example, producing pharmaceutical preparations. Thus, purified proteins may be those of an extract. The purified protein may be just one component of the compositions as provided herein, and thus, the protein may be only a small percentage by weight of the composition. The purified protein is nonetheless purified in that it is separate or has been separated from substances, such as substances with which it may be associated in a naturally-available raw material. In some embodiments, however, the purified protein may be recombinantly produced or synthesized. The synthetic nanocarriers provided herein may encapsulate a purified protein or a mixture of purified proteins. When a mixture, each purified protein is as defined herein.

“Selecting” refers to making a choice or selection. The selection or choice may be based on desired properties and/or expected outcome(s). In some embodiments, synthetic nanocarriers are selected or chosen in order to practice the invention provided herein based on any one or more of the properties and/or desired outcome(s) provided herein. This term is intended to include “causing to select”. “Causing to select” means causing, urging, encouraging, aiding, inducing or directing or acting in coordination with an entity for the entity to make a selection. In some embodiments of any one of the methods provided herein, the method may comprise or further comprise any one of the steps of selecting as described herein.

“Subject” means animals, including warm blooded mammals such as humans and primates; avians; domestic household or farm animals such as cats, dogs, sheep, goats, cattle, horses and pigs; laboratory animals such as mice, rats and guinea pigs; fish; reptiles; zoo and wild animals; and the like.

“Synthetic nanocarrier(s)” means a discrete object that is not found in nature, and that possesses at least one dimension that is less than or equal to 5 microns in size. As provided herein the synthetic nanocarriers are made up, at least in part, of a polyester polymer matrix with the desired HLB values as provided herein and comprise a desirable allergen and/or load of allergen(s). Synthetic nanocarriers may be a variety of different shapes, including but not limited to spheroidal, cuboidal, pyramidal, oblong, cylindrical, toroidal, and the like. Synthetic nanocarriers according to the invention comprise one or more surfaces. In embodiments, synthetic nanocarriers may possess an aspect ratio greater than 1:1, 1:1.2, 1:1.5, 1:2, 1:3, 1:5, 1:7, or greater than 1:10.

In an embodiment of any one of the compositions provided herein, the synthetic nanocarriers that have a minimum dimension of equal to or less than about 100 nm, preferably equal to or less than 100 nm, do not comprise a surface with hydroxyl groups that activate complement or alternatively comprise a surface that consists essentially of moieties that are not hydroxyl groups that activate complement. In a preferred embodiment, synthetic nanocarriers according to the invention that have a minimum dimension of equal to or less than about 100 nm, preferably equal to or less than 100 nm, do not comprise a surface that substantially activates complement or alternatively comprise a surface that consists essentially of moieties that do not substantially activate complement. In a more preferred embodiment, synthetic nanocarriers according to the invention that have a minimum dimension of equal to or less than about 100 nm, preferably equal to or less than 100 nm, do not comprise a surface that activates complement or alternatively comprise a surface that consists essentially of moieties that do not activate complement.

“Weight %” or “wt %” refers to the ratio of one weight to another weight times 100. For example, the weight % can be the ratio of the weight of one component to another times 100 or the ratio of the weight of one component to a total weight of more than one component times 100. Generally, the weight %, when referring to synthetic nanocarriers, is measured as an average across a population of synthetic nanocarriers or an average across the synthetic nanocarriers in a composition or suspension.

“Weighted mean retention time of a sample of an allergen” refers to the weighted mean as determined by Equation 3 using a reverse-phase high performance liquid chromatography (RP-HPLC) system. The RP-HPLC may be any one as described in the Examples, and the method of determining this measure may be performed with any one of such methods provided in the Examples. In some embodiments of any one of the compositions or methods provided herein, this weighted mean retention time is between 1 and 10 minutes. In some embodiments of any one of the compositions or methods provided herein, this weighted mean retention time of a sample of the allergen is between 2 and 10 minutes, between 3 and 10 minutes, between 4 and 10 minutes, between 5 and 10 minutes, between 6 and 10 minutes, between 7 and 10 minutes, between 8 and 10 minutes or between 9 and 10 minutes. In some embodiments of any one of the compositions or methods provided herein, this weighted mean retention time of a sample of the allergen is between 2 and 9 minutes, between 3 and 9 minutes, between 4 and 9 minutes, between 5 and 9 minutes, between 6 and 9 minutes, between 7 and 9 minutes or between 8 and 9 minutes. In some embodiments of any one of the compositions or methods provided herein, this weighted mean retention time of a sample of the allergen is between 2 and 8 minutes, between 3 and 8 minutes, between 4 and 8 minutes, between 5 and 8 minutes, between 6 and 8 minutes or between 7 and 8 minutes. In some embodiments of any one of the compositions or methods provided herein, this weighted mean retention time of a sample of the allergen is between 2 and 7 minutes, between 3 and 7 minutes, between 4 and 7 minutes, between 5 and 7 minutes or between 6 and 7 minutes. In some embodiments of any one of the compositions or methods provided herein, this weighted mean retention time of a sample of the allergen is between 2 and 6 minutes, between 3 and 6 minutes, between 4 and 6 minutes or between 5 and 6 minutes. In some embodiments of any one of the compositions or methods provided herein, this weighted mean retention time of a sample of the allergen is between 2 and 5 minutes, between 3 and 5 minutes or between 4 and 5 minutes. In some embodiments of any one of the compositions or methods provided herein, this weighted mean retention time of a sample of the allergen is between 2 and 4 minutes or between 3 and 4 minutes. In some embodiments of any one of the compositions or methods provided herein, this weighted mean retention time of a sample of the allergen is between 2 and 3 minutes. A “sample of the allergen” as used herein refers to a portion of a composition comprising the allergen used in producing the synthetic nanocarriers as provided herein or a composition comprising the allergen that is considered comparable to such a composition comprising the allergen or portion thereof.

### C. Compositions and Related Methods

Provided herein are synthetic nanocarrier compositions that are optimized to encapsulate allergens at a substantial load while minimizing the amount of allergen present on the nanocarrier surface. This can be helpful to avoid unwanted allergic responses that can be characterized by Th2 response-biased cytokines. Specifically, it has been unexpectedly discovered that the relative hydrophobicity of the polymer matrix on which the structure of the synthetic nanocarrier is based, measured as HLB, can affect the amount of allergen on the surface of the nanocarriers as well as the amount incorporated within the synthetic nanocarriers. In other words, there is a specific range of relative hydrophobicity of the polymer matrix that can result in minimal surface display of the allergens with substantial encapsulation. Also, in some embodiments, a measure of allergen hydrophobicity can also be important.

Without wishing to be bound by any particular theory, a reduction in the surface display of allergen is expected to minimize the allergen exposure to a subject until the nanocarriers reach their site of action. Using Griffin's method (Griffin 1949 and Griffin 1954), to calculate the relative hydrophilic to lipophilic balance (HLB), of the polymer matrix, it was observed that polymer mixtures which are too hydrophilic or too hydrophobic tend to display more of the allergen on the surface or result in allergen loss during synthetic nanocarrier formulation. Ideal polymer matrices were found to have calculated HLB values within the range of 11 to 15. Thus, optimized synthetic nanocarriers provided herein can comprise polyester polymer matrices that have an HLB ranging from 11 to 15. In some embodiments of any one of the compositions or methods provided herein, the HLB is from 11 to 14, 11 to 13.9, 11 to 13.8, 11 to 13.7, 11 to 13.6, 11 to 13.5, 11 to 13.4, 11 to 13.3, 11 to 13.2, 11 to 13.1 or 11 to 13. In other embodiments of any one of the compositions or methods provided herein, the HLB is from 11 to 12. In still other embodiments of any one of the compositions or methods provided herein, the HLB is from 11.5 to 14, from 11.5 to 13, 11.5 to 12, 12 to 13, 12 to 14 or 13 to 14. In still other embodiments of any one of the compositions or methods provided herein, the HLB is from 12 to 14.5, 12 to 14, 12 to 13.5 or 12 to 13. In other embodiments of any one of the compositions or methods provided herein, the HLB is from 12.5 to 14.5, 12.5 to 14, 12.5 to 13.5 or 12.5 to 13. In yet other embodiments of any one of the compositions or methods provided herein, the HLB is 11, 11.5, 12, 12.5, 13, 13.5, 14, 14.5 or 15. The synthetic nanocarriers with such matrices can be used to encapsulate allergens at a substantial load. Examples of allergen are provided elsewhere herein.

As mentioned above, the physiochemical properties of the allergens may also play a role in the production of the synthetic nanocarriers as provided herein and/or the in vivo effects of use of such synthetic nanocarriers. In order to characterize a desirable feature of the allergens for use herein, a measure of relative hydrophobicity of the allergens may be calculated. In some embodiments of any one of the compositions or methods provided herein, this measure is the weighted mean retention time as calculated by Equation 3 using a RP-HPLC system. The RP-HPLC system may be any one of such systems provided herein. The method for determining the weighted mean retention time for any one of the compositions or methods provided herein may be any one of such methods provided herein.

For example, in one embodiment of any one of the compositions or methods provided, the RP-HPLC can be performed on an ultra high pressure liquid chromatography instrument (UHPLC), such as an Agilent UHPLC instrument. In one embodiment of any one of the compositions or methods provided, the column can be a C18 UHPLC column, such as an XBridge Peptide BEH C18 UHPLC column. The sample of the allergen can be monitored at 200 nm absorbance with a flow-rate of 3.0 mL/minute with the sample of the allergen injected onto the column containing 3 μg of allergen. In one embodiment of any one of the compositions or methods provided, the mobile phase A of the RP-HPLC can be composed of 94.9% water, 5% acetonitrile, and 0.1% trifluoroacetic acid on a volume percent basis, and the mobile phase B of the RP-HPLC can be composed of 19.9% water, 80% acetonitrile, 0.1% trifluoroacetic acid on a volume percent basis. The water for the system can be supplied by a reverse osmosis deionized water system and 0.2 μm filtered.

The weighted mean retention time can be calculated according to any one of the methods provided herein or otherwise known in the art. In one embodiment of any one of the compositions or methods provided, the protein peaks can be identified, and the area under the curve can be calculated using chromatography software. In one embodiment of any one of the compositions or methods provided, the weight percent of each peak can be calculated based on the area under the curve and can be divided by the sum of the area under the peaks for all identified protein peaks. In one embodiment of any one of the compositions or methods provided, the chromatography software can be Agilent chromatography software.

The loads of allergen in the synthetic nanocarriers provided herein can be substantial and have also been determined. In some embodiments of any one of the compositions or methods provided herein, the synthetic nanocarriers on average across a population of synthetic nanocarriers comprise 0.5 to 2.5 wt % allergen to total synthetic nanocarrier materials. In other embodiments of any one of the compositions or methods provided herein, the load of the allergen is 0.5 to 2, 0.5 to 1.5 or 0.5 to 1 wt % allergen to total synthetic nanocarrier materials. In still other embodiments of any one of the compositions or methods provided herein, the load of the allergen is 0.75 to 2.5, 0.75 to 2, 0.75 to 1.5 or 0.75 to 1 wt % to total synthetic nanocarrier materials. In still other embodiments of any one of the compositions or methods provided, the load of the allergen is 1 to 1.5, 1 to 2 or 1 to 2.5 wt % to total synthetic nanocarrier materials. In further embodiments of any one of the compositions or methods provided herein, the load of the allergen is 0.5, 0.75, 1, 1.25, 1.5, 1.75, 2 or 2.5 wt % to total synthetic nanocarrier materials. As used herein, the amount of the allergen is the total amount of the allergen material whether the allergen is one or more types of allergen (such as when the allergen is in the form of a mixture of purified proteins or an extract in the synthetic nanocarriers).

The polyester polymer matrices comprise at least one type of polyester. Polyesters include copolymers comprising lactic acid and glycolic acid units, such as poly(lactic acid-co-glycolic acid) and poly(lactide-co-glycolide), collectively referred to herein as “PLGA”; and homopolymers comprising glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA.” In some embodiments, exemplary polyesters include, for example, polyhydroxyacids; PEG copolymers and copolymers of lactide and glycolide (e.g., PLA-PEG copolymers, PGA-PEG copolymers, PLGA-PEG copolymers, and derivatives thereof. In some embodiments, polyesters include, for example, poly(caprolactone), poly(caprolactone)-PEG copolymers, poly(L-lactide-co-L-lysine), poly(serine ester), poly(4-hydroxy-L-proline ester), poly[α-(4-aminobutyl)-L-glycolic acid], and derivatives thereof.

In some embodiments, a polymer of the polyester polymer matrix may be PLGA. PLGA is a biocompatible and biodegradable co-polymer of lactic acid and glycolic acid, and various forms of PLGA are characterized by the ratio of lactic acid:glycolic acid. Lactic acid can be L-lactic acid, D-lactic acid, or D,L-lactic acid. The degradation rate of PLGA can be adjusted by altering the lactic acid:glycolic acid ratio. In some embodiments, PLGA to be used in accordance with the present invention is characterized by a lactic acid:glycolic acid ratio of approximately 85:15, approximately 75:25, approximately 60:40, approximately 50:50, approximately 40:60, approximately 25:75, or approximately 15:85.

The polyester polymer matrices may also comprise one or more other polymers, provided that the matrix of polymers satisfy the HLB criteria as provided herein. Such other polymers may also be polyesters, functionalized or derivatized polyesters, or a copolymer comprising a polyester, such as a copolymer of a polyester and a hydrophilic polymer, such as polyethylene glycol, a peptide polymer, polyacrylic acid, etc. In one embodiment of any one of the compositions or methods provided, the copolymer comprising a polyester may also comprise a polyether, such as polyethylene glycol. Optimal amounts of the polymers of such copolymers have also been determined for some embodiments. For example, in one embodiment of any one of the compositions provided, when the block copolymer comprises PLGA and PEG, the wt % of the PEG is no more than 5 wt %. In some embodiments, when the block copolymer comprises PLA and PEG, the wt % is no more than 4.5, 4.25, 4 or 3.75 wt %. As another example, in one embodiment of any one of the compositions provided, when the block copolymer comprises PLA and PEG, the wt % of the PEG is from 0 to 15 wt %. In some embodiments, when the block copolymer comprises PLA and PEG, the wt % is from 0 to 14.5, 0 to 14, 0 to 13.5 or 1 to 13.5 wt %.

The one or more other polymers may be a non-methoxy-terminated, pluronic polymer, or a unit thereof. “Non-methoxy-terminated polymer” means a polymer that has at least one terminus that ends with a moiety other than methoxy. In some embodiments, the polymer has at least two termini that ends with a moiety other than methoxy. In other embodiments, the polymer has no termini that ends with methoxy. “Non-methoxy-terminated, pluronic polymer” means a polymer other than a linear pluronic polymer with methoxy at both termini.

In some embodiments, the one or more other polymers may be polyhydroxyalkanoates, polyamides, polyethers, polyolefins, polyacrylates, polycarbonates, polystyrene, silicones, fluoropolymers, or a unit thereof. Further examples of polymers that may be comprised in the polyester polymer matrices provided herein include polycarbonate, polyamide, or polyether, or unit thereof. In other embodiments, the polymers of the polyester polymer matrices may comprise polyethylene glycol, or a unit thereof.

In some embodiments, it is preferred that the polyester polymer matrix comprises polymer that is biodegradable. In some embodiments, polymers in accordance with the present invention include polymers which have been approved for use in humans by the U.S. Food and Drug Administration (FDA) under 21 C.F.R. § 177.2600. Therefore, in such embodiments, the polymers of the polyester polymer matrix may include a polyether, such as poly(ethylene glycol) or unit thereof. Additionally, the polymer may comprise a block-co-polymer of a polyether and a biodegradable polymer such that the polymer is biodegradable. In other embodiments, the polymer does not solely comprise a polyether or unit thereof, such as poly(ethylene glycol) or unit thereof.

Other examples of polymers suitable for use in the present invention include, but are not limited to polyethylenes, polycarbonates (e.g. poly(1,3-dioxan-2one)), polyanhydrides (e.g. poly(sebacic anhydride)), polypropylfumerates, polyamides (e.g. polycaprolactam), polyacetals, polyethers, polyesters (e.g., polylactide, polyglycolide, polylactide-co-glycolide, polycaprolactone, polyhydroxyacid (e.g. poly(β-hydroxyalkanoate))), poly(orthoesters), polycyanoacrylates, polyvinyl alcohols, polyurethanes, polyphosphazenes, polyacrylates, polymethacrylates, polyureas, polystyrenes, and polyamines, polylysine, polylysine-PEG copolymers, and poly(ethyleneimine), poly(ethylene imine)-PEG copolymers.

Still other examples of polymers that may be included in a polyester polymer matrix include acrylic polymers, for example, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, polycyanoacrylates, and combinations comprising one or more of the foregoing polymers.

In some embodiments, polymers may be modified with one or more moieties and/or functional groups. A variety of moieties or functional groups can be used in accordance with the present invention. In some embodiments, polymers may be modified with polyethylene glycol (PEG), with a carbohydrate, and/or with acyclic polyacetals derived from polysaccharides (Papisov, 2001, ACS Symposium Series, 786:301). Certain embodiments may be made using the general teachings of U.S. Pat. No. 5,543,158 to Gref et al., or WO publication WO2009/051837 by Von Andrian et al.

In some embodiments, polymers may be modified with a lipid or fatty acid group. In some embodiments, a fatty acid group may be one or more of butyric, caproic, caprylic, capric, lauric, myristic, palmitic, stearic, arachidic, behenic, or lignoceric acid. In some embodiments, a fatty acid group may be one or more of palmitoleic, oleic, vaccenic, linoleic, alpha-linoleic, gamma-linoleic, arachidonic, gadoleic, arachidonic, eicosapentaenoic, docosahexaenoic, or erucic acid.

Polymers may be natural or unnatural (synthetic) polymers. Polymers may be homopolymers or copolymers comprising two or more monomers. In terms of sequence, copolymers may be random, block, or comprise a combination of random and block sequences.

In some embodiments, polymers can be linear or branched polymers. In some embodiments, polymers can be dendrimers. In some embodiments, polymers can be substantially cross-linked to one another. In some embodiments, polymers can be substantially free of cross-links. In some embodiments, polymers can be used in accordance with the present invention without undergoing a cross-linking step. It is further to be understood that the synthetic nanocarriers may comprise block copolymers, graft copolymers, blends, mixtures, and/or adducts of any of the foregoing and other polymers. Those skilled in the art will recognize that the polymers listed herein represent an exemplary, not comprehensive, list of polymers that can be of use in accordance with the present invention provided they meet the desired criteria.

The properties of these and other polymers and methods for preparing them are well known in the art (see, for example, U.S. Pat. Nos. 6,123,727; 5,804,178; 5,770,417; 5,736,372; 5,716,404; 6,095,148; 5,837,752; 5,902,599; 5,696,175; 5,514,378; 5,512,600; 5,399,665; 5,019,379; 5,010,167; 4,806,621; 4,638,045; and U.S. Pat. No. 4,946,929; Wang et al., 2001, J. Am. Chem. Soc., 123:9480; Lim et al., 2001, J. Am. Chem. Soc., 123:2460; Langer, 2000, Acc. Chem. Res., 33:94; Langer, 1999, J. Control. Release, 62:7; and Uhrich et al., 1999, Chem. Rev., 99:3181). More generally, a variety of methods for synthesizing certain suitable polymers are described in Concise Encyclopedia of Polymer Science and Polymeric Amines and Ammonium Salts, Ed. by Goethals, Pergamon Press, 1980; Principles of Polymerization by Odian, John Wiley & Sons, Fourth Edition, 2004; Contemporary Polymer Chemistry by Allcock et al., Prentice-Hall, 1981; Deming et al., 1997, Nature, 390:386; and in U.S. Pat. Nos. 6,506,577, 6,632,922, 6,686,446, and 6,818,732.

The amounts of components or materials as recited herein for any one of the compositions or methods provided herein can be determined using methods known to those of ordinary skill in the art or otherwise provided herein. For example, amounts can be measured by extraction followed by quantitation by an HPLC method. Amounts of polymer can be determined using HPLC. The determination of such an amount may, in some embodiments, follow the use of proton NMR or other orthogonal methods, such as MALDI-MS, etc. to determine the identity of a polymer. Similar methods can be used to determine the amounts of allergen in any one of the compositions provided herein. For any one of the compositions or methods provided herein the amounts of the components or materials can also be determined based on the recipe weights of a nanocarrier formulation. Accordingly, in some embodiments of any one of the compositions or methods provided herein, the amounts of any one of the components provided herein are those of the components in an aqueous phase during formulation of the synthetic nanocarriers. In some embodiments of any one of the compositions or methods provided herein, the amounts of any one of the components are those of the components in a synthetic nanocarrier composition that is produced and the result of a formulation process.

A wide variety of synthetic nanocarriers can be used according to the invention. In some embodiments, synthetic nanocarriers are spheres or spheroids. In some embodiments, synthetic nanocarriers are flat or plate-shaped. In some embodiments, synthetic nanocarriers are cubes or cubic. In some embodiments, synthetic nanocarriers are ovals or ellipses. In some embodiments, synthetic nanocarriers are cylinders, cones, or pyramids.

In some embodiments, it is desirable to use a population of synthetic nanocarriers that is relatively uniform in terms of size or shape so that each synthetic nanocarrier has similar properties. For example, at least 80%, at least 90%, or at least 95% of the synthetic nanocarriers, based on the total number of synthetic nanocarriers, may have a minimum dimension or maximum dimension that falls within 5%, 10%, or 20% of the average diameter or average dimension of the synthetic nanocarriers.

Compositions according to the invention can comprise elements in combination with pharmaceutically acceptable excipients, such as preservatives, buffers, saline, or phosphate buffered saline. The compositions may be made using conventional pharmaceutical manufacturing and compounding techniques to arrive at useful dosage forms. In an embodiment, compositions, such as those comprising the synthetic nanocarriers are suspended in sterile saline solution for injection together with a preservative.

### D. Methods of Making and Using the Compositions and Related Methods

Synthetic nanocarriers may be prepared using a wide variety of methods known in the art. For example, synthetic nanocarriers can be formed by methods such as nanoprecipitation, flow focusing using fluidic channels, spray drying, single and double emulsion solvent evaporation, solvent extraction, phase separation, milling (including cryomilling), supercritical fluid (such as supercritical carbon dioxide) processing, microemulsion procedures, microfabrication, nanofabrication, sacrificial layers, simple and complex coacervation, and other methods well known to those of ordinary skill in the art. Alternatively or additionally, aqueous and organic solvent syntheses for monodisperse semiconductor, conductive, magnetic, organic, and other nanomaterials have been described (Pellegrino et al., 2005, Small, 1:48; Murray et al., 2000, Ann. Rev. Mat. Sci., 30:545; and Trindade et al., 2001, Chem. Mat., 13:3843). Additional methods have been described in the literature (see, e.g., Doubrow, Ed., “Microcapsules and Nanoparticles in Medicine and Pharmacy,” CRC Press, Boca Raton, 1992; Mathiowitz et al., 1987, J. Control. Release, 5:13; Mathiowitz et al., 1987, Reactive Polymers, 6:275; and Mathiowitz et al., 1988, J. Appl. Polymer Sci., 35:755; U.S. Pat. Nos. 5,578,325 and 6,007,845; P. Paolicelli et al., “Surface-modified PLGA-based Nanoparticles that can Efficiently Associate and Deliver Virus-like Particles” Nanomedicine. 5(6):843-853 (2010)).

Various materials may be encapsulated into synthetic nanocarriers as desirable using a variety of methods including but not limited to C. Astete et al., “Synthesis and characterization of PLGA nanoparticles” J. Biomater. Sci. Polymer Edn, Vol. 17, No. 3, pp. 247-289 (2006); K. Avgoustakis “Pegylated Poly(Lactide) and Poly(Lactide-Co-Glycolide) Nanoparticles: Preparation, Properties and Possible Applications in Drug Delivery” Current Drug Delivery 1:321-333 (2004); C. Reis et al., “Nanoencapsulation I. Methods for preparation of drug-loaded polymeric nanoparticles” Nanomedicine 2:8-21 (2006); P. Paolicelli et al., “Surface-modified PLGA-based Nanoparticles that can Efficiently Associate and Deliver Virus-like Particles” Nanomedicine. 5(6):843-853 (2010). Other methods suitable for encapsulating materials into synthetic nanocarriers may be used, including without limitation methods disclosed in U.S. Pat. No. 6,632,671 to Unger issued Oct. 14, 2003.

In certain embodiments, synthetic nanocarriers are prepared by a nanoprecipitation process or spray drying. Conditions used in preparing synthetic nanocarriers may be altered to yield particles of a desired size or property (e.g., hydrophobicity, hydrophilicity, external morphology, “stickiness,” shape, etc.). The method of preparing the synthetic nanocarriers and the conditions (e.g., solvent, temperature, concentration, air flow rate, etc.) used may depend on the materials to be included in the synthetic nanocarriers and/or the composition of the carrier matrix.

If synthetic nanocarriers prepared by any of the above methods have a size range outside of the desired range, such synthetic nanocarriers can be sized, for example, using a sieve.

Compositions provided herein may comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol).

Compositions according to the invention may comprise pharmaceutically acceptable excipients. The compositions may be made using conventional pharmaceutical manufacturing and compounding techniques to arrive at useful dosage forms. Techniques suitable for use in practicing the present invention may be found in Handbook of Industrial Mixing: Science and Practice, Edited by Edward L. Paul, Victor A. Atiemo-Obeng, and Suzanne M. Kresta, 2004 John Wiley & Sons, Inc.; and Pharmaceutics: The Science of Dosage Form Design, 2nd Ed. Edited by M. E. Auten, 2001, Churchill Livingstone. In an embodiment, compositions are suspended in a sterile saline solution for injection together with a preservative.

It is to be understood that the compositions of the invention can be made in any suitable manner, and the invention is in no way limited to compositions that can be produced using the methods described herein. Selection of an appropriate method of manufacture may require attention to the properties of the particular elements being associated.

In some embodiments, compositions are manufactured under sterile conditions or are initially or terminally sterilized. This can ensure that resulting compositions are sterile and non-infectious, thus improving safety when compared to non-sterile compositions. This provides a valuable safety measure, especially when subjects receiving the compositions have immune defects, are suffering from infection, and/or are susceptible to infection. In some embodiments, the compositions may be lyophilized and stored in suspension or as lyophilized powder depending on the formulation strategy for extended periods without losing activity.

Administration according to the present invention may be by a variety of routes, including but not limited to intradermal, intramuscular, subcutaneous, intravenous, and intraperitoneal routes. The compositions referred to herein may be manufactured and prepared for administration using conventional methods.

The compositions of the invention can be administered in effective amounts, such as the effective amounts described elsewhere herein. Doses of dosage forms may contain varying amounts of elements according to the invention. The amount of elements present in the inventive dosage forms can be varied according to their nature, the therapeutic benefit to be accomplished, and other such parameters. In embodiments, dose ranging studies can be conducted to establish optimal therapeutic amounts to be present in the dosage form. In embodiments, the elements are present in the dosage form in an amount effective to generate a desired effect and/or immune response upon administration to a subject. It may be possible to determine amounts to achieve a desired result using conventional dose ranging studies and techniques in subjects. Inventive dosage forms may be administered at a variety of frequencies. In an embodiment, at least one administration of the compositions provided herein is sufficient to generate a pharmacologically relevant response.

Another aspect of the disclosure relates to kits. In some embodiments, the kit comprises any one of the compositions provided herein. In some embodiments of any one of the kits provided, the kit comprises any one or more of the allergens provided herein and any one of the polyester polymer matrices as provided herein. In some embodiments of any one of the kits provided, the kit further comprises an adjuvant, such as a Th1-biasing adjuvant. In some embodiments of any one of the kits provided, the compositions or elements thereof can be contained within separate containers or within the same container in the kit. In some embodiments of any one of the kits provided, the container is a vial or an ampoule. In some embodiments of any one of the kits provided, the compositions or elements thereof are contained within a solution separate from the container, such that the compositions or elements may be added to the container at a subsequent time. In some embodiments of any one of the kits provided, the compositions or elements thereof are in lyophilized form each in a separate container or in the same container, such that they may be reconstituted at a subsequent time. In some embodiments of any one of the kits provided, the kit further comprises instructions for reconstitution, mixing, administration, etc. In some embodiments of any one of the kits provided, the instructions include a description of the methods described herein. Instructions can be in any suitable form, e.g., as a printed insert or a label. In some embodiments of any one of the kits provided herein, the kit further comprises one or more syringes or other device(s) that can deliver any one of the compositions provided, such as a synthetic nanocarrier composition, in vivo to a subject.

### EXAMPLES

### Example 1: Synthetic Nanocarriers

### Materials

100 DL mPEG 5000 (15 wt % PEG), polylactide block co-polymer with a methyl ether terminated PEG block of approximately 5,000 Da and an overall inherent viscosity of 0.50 DL/g was purchased from Evonik Industries (Rellinghauser Straβe 1-11 45128 Essen, Germany), product code 100 DL mPEG 5000 (15 wt % PEG).

100 DL mPEG 5000 4CE polylactide block co-polymer with a methyl ether terminated PEG block of approximately 5,000 Da and an overall inherent viscosity of 0.36 DL/g was purchased from Evonik Industries (Rellinghauser Straβe 1-11 45128 Essen, Germany), product code 100 DL mPEG 5000 4CE.

100 DL mPEG 5000 (54 wt % PEG) polylactide block co-polymer with a methyl ether terminated PEG block of approximately 5,000 Da and an overall inherent viscosity of 0.22 DL/g was purchased from Evonik Industries (Rellinghauser Straβe 1-11 45128 Essen, Germany), product code 100 DL mPEG 5000 (54 wt % PEG).

PLA (D,L-lactide), of approximately 14,000 Da with an inherent viscosity of 0.21 dL/g was purchased from Evonik Industries (Rellinghauser Straβe 1-11 45128 Essen, Germany), product code R 202 H.

PLGA (poly(lactic-co-glycolic acid)), composed of 51% lactide and 49% glycolide of approximately 25,000 Da with an inherent viscosity of 0.21 dL/g was purchased from Evonik Industries (Rellinghauser Straβe 1-11 45128 Essen Germany), product code RG 502 H.

PVL (poly(delta-valerolactone) acid endcap), Mw of approximately 110,000 Da was purchased from Polyscitech (3495 Kent Avenue, West Lafayette Ind. 47906), product code AP115.

PCL (polycaprolactone), Mw of approximately 14,000 Da with an inherent viscosity of 731 MPa was purchased from Sigma-Aldrich (3050 Spruce St. St. Louis, Mo. 63103), product code 440752.

Dichloromethane was purchased from Spectrum (14422 S San Pedro Gardena Calif., 90248-2027). Part number M1266.

Ovalbumin was purchased from Worthington Biochemical Corporation (730 Vassar Ave. Lakewood, N.J. 08701), product code LS003054.

EMPROVE® Polyvinyl Alcohol 4-88, USP (85-89% hydrolyzed, viscosity of 3.4-4.6 mPa·s) was purchased from EMD Chemicals Inc. (480 South Democrat Road Gibbstown, N.J. 08027), product code 1.41350.

Phosphate-Buffered Saline (PBS) 1×: solution without Calcium and Magnesium was purchased from Corning Inc. (One Riverfront Plaza Corning, N.Y. 14831), part number 21-040.

Emulsification was carried out using a Branson Digital Sonifier 250 with a ⅛″ tapered tip titanium probe.

### Method

Solutions were prepared as follows:

Solution 1: Polymer solution were prepared by dissolving 100 mg of core polymer per 1 mL dichloromethane, for a subset of lots. For the other lots, polymer solutions were prepared by dissolving 75 mg of the indicated core polymer with 25 mg of the indicated block-copolymer per 1 mL in dichloromethane.

Solution 2: Ovalbumin was dissolved at 5 mg per 1 mL in PBS.

Solution 3: A polyvinyl alcohol solution was prepared by dissolving polyvinyl alcohol (EMPROVE® Polyvinyl Alcohol 4-88) at 75 mg per mL in 100 mM phosphate buffer pH 8.

An O/W emulsion was prepared by combining 1 mL Solution 1 and 0.2 mL Solution 2 in a small glass pressure tube. The solution was mixed by repeat pipetting. The formulation was then sonicated with the pressure tube immersed in an ice water bath for 40 seconds at 50% amplitude. Next, a W/O/W emulsion was prepared by adding Solution 3 (3 mL), to the glass pressure tube. The tube was vortex mixed for ten seconds, then the formulation was sonicated with the pressure tube immersed in an ice bath for 1 minute at 30% amplitude. The emulsion was then added to an open 50 mL beaker containing PBS (30 mL). This was then stirred at room temperature for 2 hours to allow the dichloromethane to evaporate and for the nanocarriers to form. A portion of the nanocarriers were washed by transferring the nanocarrier suspension to a centrifuge tube and centrifuging at 75,600 g at 4° C. for 50 minutes, removing the supernatant, and re-suspending the pellet in PBS. The wash procedure was repeated and then the pellet was re-suspended in PBS to achieve a nanocarrier suspension having a nominal concentration of 10 mg per mL on a polymer basis. The nanocarrier formulation was filtered using a 0.22 μm syringe filter (EMD Millipore part number SLGP033RS), and/or a 0.45 μm syringe filter (Pall Corporation, part number 4654). The filtered nanocarrier solution was then stored at −20° C.

Nanocarrier size was determined by dynamic light scattering. Ovalbumin load was determined using a quantitative plate assay. The nanocarrier yield was determined by a gravimetric method. Surface antigen presentation was determined by ELISA.

TABLE 1
Formulations prepared with ovalbumin

| | | | Ag | OVA |
|---|---|---|---|---|
| Core | | Size | Load | ELISA |
| Polymer | Block Co-Polymer | (nm) | (%) | Top OD |
| PLGA | — | 122 | 0.62 | 0.566 |
| PLGA | 100 DL mPEG 5000 (15 wt % PEG) | 118 | 0.94 | 0.750 |
| PLGA | 100 DL mPEG 5000 4CE | 112 | 0.87 | 0.885 |
| PLGA | 100 DL mPEG 5000 (54 wt % PEG) | 91 | 0.97 | 0.981 |
| PLA | — | 123 | 0.75 | 0.533 |
| PLA | 100 DL mPEG 5000 (15 wt % PEG) | 134 | 1.28 | 0.588 |
| PLA | 100 DL mPEG 5000 4CE | 128 | 0.84 | 0.586 |
| PLA | 100 DL mPEG 5000 (54 wt % PEG) | 113 | 0.87 | 0.655 |
| PVL | — | 170 | 1.42 | 0.874 |
| PVL | 100 DL mPEG 5000 (15 wt % PEG) | 163 | 0.84 | 0.965 |
| PVL | 100 DL mPEG 5000 4CE | 155 | 0.81 | 0.867 |
| PVL | 100 DL mPEG 5000 (54 wt % PEG) | 145 | 0.65 | 0.827 |
| PCL | — | 163 | 0.09 | 0.735 |
| PCL | 100 DL mPEG 5000 (15 wt % PEG) | 156 | 0.72 | 1.099 |
| PCL | 100 DL mPEG 5000 4CE | 155 | 0.64 | 1.064 |
| PCL | 100 DL mPEG 5000 (54 wt % PEG) | 149 | 0.22 | 1.042 |

### Materials

PLA-PEG-OMe, poly(D,L-lactide) block co-polymer with a methyl ether terminated PEG block of approximately 5,000 Da and an overall inherent viscosity of 0.50 dL/g was purchased from Evonik Industries (Rellinghauser Straβe 1-11 45128 Essen, Germany), product code 100 DL mPEG 5000 (15 wt % PEG).

PLGA (poly(lactic-co-glycolic acid)), composed of 54% lactide and 46% glycolide with an inherent viscosity of 0.24 dL/g was purchased from Evonik Industries (Rellinghauser Straβe 1-11 45128 Essen Germany), product code 5050 DLG 2.5A.

Dichloromethane was purchased from Spectrum (14422 S San Pedro Gardena Calif., 90248-2027). Part number M1266.

Ovalbumin was purchased from Worthington Biochemical Corporation (730 Vassar Ave. Lakewood, N.J. 08701), product code LS003054.

EMPROVE® Polyvinyl Alcohol 4-88, USP (85-89% hydrolyzed, viscosity of 3.4-4.6 mPa·s) was purchased from EMD Chemicals Inc. (480 South Democrat Road Gibbstown, N.J. 08027), product code 1.41350.

Phosphate-Buffered Saline (PBS) 1×: solution without Calcium and Magnesium was purchased from Corning Inc. (One Riverfront Plaza Corning, N.Y. 14831), part number 21-040.

Emulsification was carried out using a Branson Digital Sonifier 250 with a ⅛″ tapered tip titanium probe.

### Method

Solutions were prepared as follows:

Solution 1: A polymer mixture was prepared by dissolving PLA-PEG-OMe and PLGA at 100 mg per 1 mL in dichloromethane at a 1:3 ratio of PLA-PEG to PLGA.

Solution 2: Ovalbumin was dissolved in PBS at the indicated mg per 1 mL.

Solution 3: A polyvinyl alcohol solution was prepared by dissolving polyvinyl alcohol (EMPROVE® Polyvinyl Alcohol 4-88) at 75 mg per mL in 100 mM phosphate buffer pH 8.

An O/W emulsion was prepared by combining 1 mL Solution 1 and Solution 2 at the indicated volume in a small glass pressure tube. The solutions were mixed by repeat pipetting. The formulation was then sonicated with the pressure tube immersed in an ice water bath for 40 seconds at 50% amplitude on an ice water bath. Next a W/O/W emulsion was prepared by adding Solution 3 (3 mL) to the glass pressure tube. The tube was vortex mixed for ten seconds, then the formulation was sonicated with the pressure tube immersed in an ice bath for 1 minute at 30% amplitude on an ice water bath. The emulsion was then added to an open 50 mL beaker containing PBS (30 mL). This was then stirred at room temperature for 2 hours to allow the dichloromethane to evaporate and for the nanocarriers to form. A portion of the nanocarriers were washed by transferring the nanocarrier suspension to a centrifuge tube and centrifuging at 75,600 g at 4° C. for 50 minutes, removing the supernatant, and re-suspending the pellet in PBS. The wash procedure was repeated and then the pellet was re-suspended in PBS to achieve a nanocarrier suspension having a nominal concentration of 10 mg per mL on a polymer basis. The nanocarrier formulation was filtered using a 0.22 μm syringe filter (EMD Millipore, part number SLGP033RS). The filtered nanocarrier solution was then stored at −20° C.

Nanocarrier size was determined by dynamic light scattering. The amount of ovalbumin in the nanocarrier was determined by a quantitative plate assay. The nanocarrier yield was determined by a gravimetric method. The surface antigen presentation was determined by ELISA.

TABLE 2
Formulations prepared with ovalbumin

| Ovalbumin W1 | | | | OVA |
|---|---|---|---|---|
| Concentration | W1 volume | Size | Load | ELISA |
| (mg/mL) | (mL) | (nm) | (%) | Top OD |
| 10 | 0.50 | 165.6 | 4.97 | 1.443 |
| 10 | 0.20 | 143.4 | 1.82 | 0.924 |
| 10 | 0.15 | 141.7 | 1.41 | 0.782 |
| 10 | 0.10 | 136.9 | 0.91 | 0.658 |
| 2 | 0.50 | 157.1 | 0.94 | 0.576 |
| 5 | 0.20 | 138.2 | 1.00 | 0.649 |
| 6.7 | 0.15 | 138.0 | 1.06 | 0.706 |

### Materials

PLGA with 54% lactide and 46% glycolide content and an inherent viscosity of 0.24 dL/g was purchased from Lakeshore Biomaterials (756 Tom Martin Drive, Birmingham, Ala. 35211), product Code 5050 DLG 2.5A.

PLA-PEG-OMe block co-polymer with a methyl ether terminated PEG block of approximately 5,000 Da and an overall inherent viscosity of 0.38 dL/g was purchased from Lakeshore Biomaterials (756 Tom Martin Drive, Birmingham, Ala. 35211), product code 100 DL mPEG 5000 4CE.

EMPROVE® Polyvinyl Alcohol 4-88, USP (85-89% hydrolyzed, viscosity of 3.4-4.6 mPa·s) was purchased from EMD Chemicals Inc. (480 South Democrat Road Gibbstown, N.J. 08027), product code 1.41350.

### Methods

Solutions were prepared as follows:

Solution 1: Polyvinyl alcohol was prepared at 100 mg/mL in endotoxin free water.

Solution 2: A polymer solution was prepared by dissolving PLGA at 75 mg/mL and PLA-PEG-OMe at 25 mg/mL in dichloromethane.

The methodology for preparing the synthetic nanocarriers can be similar to the above Examples.

Nanocarrier size was determined by dynamic light scattering. The total dry-nanocarrier mass per mL of suspension was determined by a gravimetric method.

TABLE 3
Nanocarrier characteristics

| Effective Diameter | Nanocarrier Yield |
|---|---|
| (nm) | (%) |
| 155 | 73 |

### Materials

PLA-PEG-OMe block co-polymer with a methyl ether terminated PEG block of approximately 5,000 Da and an overall inherent viscosity of 0.50 DL/g was purchased from Evonik Industries (Rellinghauser Straβe 1-11 45128 Essen, Germany), product code 100 DL mPEG 5000 (15 wt % PEG).

PLGA (poly(lactic-co-glycolic acid)), composed of 51% lactide and 49% glycolide with an inherent viscosity of 0.2 dL/g was purchased from Evonik Industries (Rellinghauser Straβe 1-11 45128 Essen Germany), product code RG 502 H.

Dichloromethane was purchased from Spectrum (14422 S San Pedro Gardena Calif., 90248-2027). Part number M1266.

Complete Peanut Extract was provided by Sanofi Pasteur (82, Avenue Raspail, Gentilly, France), lot number PS 1140223.

EMPROVE® Polyvinyl Alcohol 4-88, USP (85-89% hydrolyzed, viscosity of 3.4-4.6 mPa·s) was purchased from EMD Chemicals Inc. (480 South Democrat Road Gibbstown, N.J. 08027), product code 1.41350.

Dulbecco's phosphate buffered saline 1× (DPBS) was purchased from Lonza (Muenchensteinerstrasse 38, CH-4002 Basel, Switzerland), product code 17-512Q.

Phosphate-Buffered Saline 1× (PBS), solution without Calcium and Magnesium was purchased from Corning Inc. (One Riverfront Plaza Corning, N.Y. 14831), part number 21-040.

Emulsification was carried out using a Branson Digital Sonifier 250 with a ⅛″ tapered tip titanium probe.

### Method

Solutions were prepared as follows:

Solution 1: A polymer mixture was prepared by dissolving PLA-PEG-OMe and PLGA at 100 mg per 1 mL dichloromethane at a 1:3 ratio of PLA-PEG-OMe to PLGA.

Solution 2: CPE was prepared at 39 mg/mL in Tris buffer and 10% sucrose.

Solution 3: A polyvinyl alcohol solution was prepared by dissolving polyvinyl alcohol (EMPROVE® Polyvinyl Alcohol 4-88) at 75 mg per mL in 50 mM Tris pH 9, 125 mM NaCl.

An O/W emulsion was prepared by combining 1 mL of Solution 1 and 0.15 mL of Solution 2, in a small glass pressure tube. The solutions were mixed by repeat pipetting. The crude emulsion was then sonicated with the pressure tube immersed in an ice water bath for 40 seconds at 50% amplitude. Next, a W/O/W emulsion was prepared by adding Solution 3 (3 mL), to the glass pressure tube. The tube was vortex mixed for ten seconds, then the formulation was sonicated with the pressure tube immersed in an ice bath for 1 minute at 30% amplitude. The emulsion was then added to an open 50 mL beaker containing DPBS (30 mL). This was then stirred at room temperature for 2 hours to allow the dichloromethane to evaporate and for the nanocarriers to form. A portion of the nanocarriers were washed by transferring the nanocarrier suspension to a centrifuge tube and centrifuging at 75,600×g and 4° C. for 50 minutes, removing the supernatant, and re-suspending the pellet in PBS. The wash procedure was repeated and then the pellet was re-suspended in PBS to achieve a nanocarrier suspension having a nominal concentration of 10 mg per mL on a polymer basis. The nanocarrier formulation was filtered using a 0.22 μm syringe filter (EMD Millipore part number SLGP033RS). The filtered nanocarrier solution was then stored at −20° C.

Nanocarrier size was determined by dynamic light scattering. The CPE load was determined using a quantitative plate based assay. The nanocarrier yield was determined by a gravimetric method. The relative surface antigen was determined by ELISA.

TABLE 4
Formulations prepared with CPE

| Size | Load | | |
|---|---|---|---|
| (nm) | (%) | Ara h2 IgG Top OD | CPE IgG1 Top OD |
| 140 | 0.93 | 2.096 | 2.367 |

### Materials

PLA-PEG-OMe block co-polymer with a methyl ether terminated PEG block of approximately 5,000 Da and an overall inherent viscosity of 0.50 DL/g was purchased from Evonik Industries (Rellinghauser Straβe 1-11 45128 Essen, Germany), product code 100 DL mPEG 5000 (15 wt % PEG).

PLA-PEG-OMe block co-polymer with a methyl ether terminated PEG block of approximately 5,000 Da and an overall inherent viscosity of 0.36 DL/g was purchased from Evonik Industries (Rellinghauser Straβe 1-11 45128 Essen, Germany), product code 100 DL mPEG 5000 (25 wt % PEG).

PLA-PEG-OMe block co-polymer with a methyl ether terminated PEG block of approximately 5,000 Da and an overall inherent viscosity of 0.22 DL/g was purchased from Evonik Industries (Rellinghauser Straβe 1-11 45128 Essen, Germany), product code 100 DL mPEG 5000 (54 wt % PEG).

Polylactic acid of approximately 14,000 Da with an inherent viscosity of 0.21 dL/g was purchased from Evonik Industries (Rellinghauser Straβe 1-11 45128 Essen, Germany), product code R 202 H.

PLGA composed of 51% lactide and 49% glycolide with an inherent viscosity of 0.2 dL/g was purchased from Evonik Industries (Rellinghauser Straβe 1-11 45128 Essen Germany), product code RG 502 H.

Polycaprolactone of approximately 14,000 Da with an inherent viscosity of 731 MPa was purchased from Sigma-Aldrich (3050 Spruce St. St. Louis, Mo. 63103), product code 440752.

Dichloromethane was purchased from Spectrum (14422 S San Pedro Gardena Calif., 90248-2027). Part number M1266.

Raw wheat gliadin was prepared as described below.

EMPROVE® Polyvinyl Alcohol 4-88, USP (85-89% hydrolyzed, viscosity of 3.4-4.6 mPa·s) was purchased from EMD Chemicals Inc. (480 South Democrat Road Gibbstown, N.J. 08027), product code 1.41350.

Phosphate-Buffered Saline (PBS) 1×: solution without Calcium and Magnesium was purchased from Corning Inc. (One Riverfront Plaza Corning, N.Y. 14831), part number 21-040.

Emulsification was carried out using a Branson Digital Sonifier 250 with a ⅛″ tapered tip titanium probe.

### Method

Solutions were prepared as follows:

Polymer Solution: A polymer mixture was prepared by either dissolving 100 mg of core polymer and no block-copolymer, or dissolving 75 mg of the corresponding core polymer with 25 mg of the indicated block-copolymer per 1 mL in dichloromethane according to the Tables.

Allergen solution: Lyophilized raw wheat gliadin was diluted at the indicated mg per 1 mL (Table 1), or at 10 mg/mL (Table 2) in 50% formamide, 50 mM acetic acid, and 0.2% beta-mercaptoethanol in E-free water.

PVA Solution: A polyvinyl alcohol solution was prepared by dissolving polyvinyl alcohol (EMPROVE® Polyvinyl Alcohol 4-88) at 75 mg per mL in 0.9% NaCl.

An O/W emulsion was prepared by combining the Polymer Solution and allergen solution (Total volume 1.1-1.5 mL) in a small glass pressure tube. The solution was mixed by repeat pipetting. The formulation was then sonicated with the pressure tube immersed in an ice water bath for 40 seconds at 50% amplitude. Next a W/O/W emulsion was prepared by adding PVA Solution (3 mL) to the glass pressure tube. The tube was vortex mixed for ten seconds, then the formulation was sonicated with the pressure tube immersed in an ice bath for 1 minute at 30% amplitude. The emulsion was then added to an open 50 mL beaker containing Cellgro PBS 1× (30 mL). This was then stirred at room temperature for 2 hours to allow the dichloromethane to evaporate and for the nanocarriers to form. A portion of the nanocarriers were washed by transferring the nanocarrier suspension to a centrifuge tube and centrifuging at 75,600×g and 4° C. for 50 minutes, removing the supernatant, and re-suspending the pellet in Corning PBS 1× Solution. The wash procedure was repeated and then the pellet was re-suspended in Corning PBS 1× Solution to achieve a nanocarrier suspension having a nominal concentration of 10 mg per mL on a polymer basis. The nanocarrier formulation was filtered using a 0.22 μm PES membrane syringe filter (MilliporeSigma, part number SLGP033RS), a 0.45 μm PES membrane syringe filter (Pall Corp., part number 4654), and/or a 1.2 μm PES membrane syringe filter (Pall Corp., part number 4656). The mass of the nanocarrier solution filter throughput was measured. The filtered nanocarrier solution was then stored at −20° C.

Nanocarrier size was determined by dynamic light scattering. The amount of raw wheat gliadin in the nanocarrier was determined by micro-BCA analysis. The total dry-nanocarrier mass per mL of suspension was determined by a gravimetric method. The surface antigen presentation was determined by a raw wheat gliadin surface ELISA.

Raw wheat gliadin was prepared. Unprocessed wheat kernels were purchased from the Modern Homebrew Emporium (Cambridge Mass., USA). The kernels were ground in a food processor to a consistent flour. The flour was dispersed into 0.5 M sodium chloride, and separated by centrifugation. This wash was repeated, then the flour re-dispersed into E-free water, mixed, and separated by centrifugation. The wheat gliadin proteins (RW gliadin), were then extracted twice by dispersing the washed flour in 70% vol/vol ethanol with 50 mM acetic acid. The collected ethanol/acetic acid fraction was then concentrated under vacuum by rotary evaporator, dialyzed overnight versus 50 mM acetic acid in water, and lyophilized.

### Materials

PLA-PEG-OMe block co-polymer with a methyl ether terminated PEG block of approximately 5,000 Da and an overall inherent viscosity of 0.50 DL/g was purchased from Evonik Industries (Rellinghauser Straβe 1-11 45128 Essen, Germany), product code 100 DL mPEG 5000 (15 wt % PEG).

PLA-PEG-OMe block co-polymer with a methyl ether terminated PEG block of approximately 5,000 Da and an overall inherent viscosity of 0.36 DL/g was purchased from Evonik Industries (Rellinghauser Straβe 1-11 45128 Essen, Germany), product code 100 DL mPEG 5000 (25 wt % PEG).

PLA-PEG-OMe block co-polymer with a methyl ether terminated PEG block of approximately 5,000 Da and an overall inherent viscosity of 0.22 DL/g was purchased from Evonik Industries (Rellinghauser Straβe 1-11 45128 Essen, Germany), product code 100 DL mPEG 5000 (54 wt % PEG).

Polylactic acid of approximately 14,000 Da with an inherent viscosity of 0.21 dL/g was purchased from Evonik Industries (Rellinghauser Straβe 1-11 45128 Essen, Germany), product code R 202 H.

PLGA composed of 51% lactide and 49% glycolide with an inherent viscosity of 0.2 dL/g was purchased from Evonik Industries (Rellinghauser Straβe 1-11 45128 Essen Germany), product code RG 502 H.

Polycaprolactone of approximately 14,000 Da with an inherent viscosity of 731 MPa was purchased from Sigma-Aldrich (3050 Spruce St. St. Louis, Mo. 63103), product code 440752.

Dichloromethane was purchased from Spectrum (14422 S San Pedro Gardena Calif., 90248-2027). Part number M1266.

Short ragweed, ambrosia artemisiifolia freeze dried extract from Stallergenes Greer (40 Bernard Street—3rd Floor London WC1N 1LE, United Kingdom), product code XP56D3A25.

House Dust Mite, dermatophagoides pteronyssinus freeze dried extract from Stallergenes Greer (40 Bernard Street—3rd Floor London WC1N 1LE, United Kingdom), product code XPB 82D3A25

EMPROVE® Polyvinyl Alcohol 4-88, USP (85-89% hydrolyzed, viscosity of 3.4-4.6 mPa·s) was purchased from EMD Chemicals Inc. (480 South Democrat Road Gibbstown, N.J. 08027), product code 1.41350.

Phosphate-Buffered Saline (PBS) 1×: solution without Calcium and Magnesium was purchased from Corning Inc. (One Riverfront Plaza Corning, N.Y. 14831), part number 21-040.

Emulsification was carried out using a Branson Digital Sonifier 250 with a ⅛″ tapered tip titanium probe.

### Methods

Solutions were prepared as follows:

Polymer Solution: A polymer mixture was prepared by either dissolving 100 mg of core polymer and no block-copolymer, or dissolving 75 mg of the corresponding core polymer with 25 mg of the indicated block-copolymer per 1 mL in dichloromethane according to the Tables.

Allergen solution: Lyophilized short ragweed or house dust mite were diluted at the indicated mg per 1 mL or at 10 mg/mL in PBS 1×.

PVA Solution: A polyvinyl alcohol solution was prepared by dissolving polyvinyl alcohol (EMPROVE® Polyvinyl Alcohol 4-88) at 75 mg per mL in 100 mM phosphate buffer pH 8.

An O/W emulsion was prepared by combining the Polymer Solution and allergen solution (Total volume 1.-1.5 mL) in a small glass pressure tube. The solution was mixed by repeat pipetting. The formulation was then sonicated with the pressure tube immersed in an ice water bath for 40 seconds at 50% amplitude. Next a W/O/W emulsion was prepared by adding PVA Solution (3 mL) to the glass pressure tube. The tube was vortex mixed for ten seconds, then the formulation was sonicated with the pressure tube immersed in an ice bath for 1 minute at 30% amplitude. The emulsion was then added to an open 50 mL beaker containing Cellgro PBS 1× (30 mL). This was then stirred at room temperature for 2 hours to allow the dichloromethane to evaporate and for the nanocarriers to form. A portion of the nanocarriers were washed by transferring the nanocarrier suspension to a centrifuge tube and centrifuging at 75,600×g and 4° C. for 50 minutes, removing the supernatant, and re-suspending the pellet in Corning PBS 1× Solution. The wash procedure was repeated and then the pellet was re-suspended in Corning PBS 1× Solution to achieve a nanocarrier suspension having a nominal concentration of 10 mg per mL on a polymer basis. The nanocarrier formulation was filtered using a 0.22 μm PES membrane syringe filter (MilliporeSigma, part number SLGP033RS), a 0.45 μm PES membrane syringe filter (Pall Corp., part number 4654), and/or a 1.2 μm PES membrane syringe filter (Pall Corp., part number 4656). The mass of the nanocarrier solution filter throughput was measured. The filtered nanocarrier solution was then stored at −20° C.

Nanocarrier size was determined by dynamic light scattering. The amount of allergen in the nanocarrier was determined by micro-BCA analysis. The total dry-nanocarrier mass per mL of suspension was determined by a gravimetric method. The surface antigen presentation was determined by a ragweed or HDM surface ELISA.

### Example 2: CPE Assays

Surface antigen presentation on each lot of CPE nanocarriers was quantified by the crude peanut extract (CPE) and Arachis hypogaea allergen (Ara h2) surface assay. CPE nanocarriers were initially diluted from stock solutions to starting concentrations of 1 μg/mL (based on CPE content) in 1×PBS. Plates were coated overnight with each nanocarriers serially diluted three fold down the plate. This was performed in two replicates for each set of CPE nanocarriers, with one replicate for CPE detection and the second replicate for Ara h2 detection. On the following day, plates were washed to remove any unbound nanocarriers and blocked with casein in PBS. The CPE serum pool was diluted 1/100, while the anti-Ara h2 antibody was diluted to 1/1,000. Following another wash step, each series of diluted nanocarriers was probed separately with diluted CPE serum pool and with anti-Ara h2 antibody. Succeeding another incubation, the plates were washed to remove any unbound antibodies, after which secondary antibodies conjugated to HRP were added to detect any CPE or Ara h2. For the CPE detection, an anti-mouse IgG1 secondary antibody diluted to 1/12,000 was applied to the microplate coated nanocarriers that had been treated with CPE serum pool, while anti-mouse IgG secondary antibody diluted to 1/1,500 was applied to the microplate coated nanocarriers that had been treated with anti-Ara h2 antibody. After another incubation and wash procedure, the presence of CPE and Ara h2 was quantified by adding TMB substrate and measuring at an absorbance of 450 nm with a reference wavelength of 570 nm. The intensity of the signal was directly proportional to the quantity of CPE and Ara h2 bound to the surface of the nanocarriers.

### Example 3: OVA/PEG Assays

Plates were coated overnight with each nanocarrier serially diluted three fold down the plate. On the following day, plates were washed to remove any unbound nanocarriers and blocked with casein to reduce non-specific binding. Following another wash step, diluted anti-OVA or anti-PEG antibodies were added to the plate and incubated. The plates were washed again to remove any unbound sample and two secondary antibodies conjugated to HRP were added to detect any OVA or PEG that bound to the nanocarrier coated plate. After another incubation and wash procedure, the presence of OVA or PEG was detected by adding TMB substrate and measuring at an absorbance of 450 nm with a reference wavelength of 570 nm. The intensity of the signal was directly proportional to the quantity of OVA or PEG bound to the surface of the nanocarriers.

### Example 4: HLB Calculations

HLB can be calculated from a known molecular structure by determining the ratio of the hydrophilic molecular weight fraction versus the total molecular weight and multiplying by 20, as described by Griffin's method (Equation 1). Using proton nuclear magnetic resonance spectroscopy (H-NMR), the Mn and structure of polyester polymers that were used in nanocarrier formulations were obtained. Using the hydrophilic versus hydrophobic molecular definitions for polymers from the literature, an unambiguous HLB value was calculated. For polymer mixtures (matrix), the combined HLB was determined by multiplying the contribution of each individual polymer by its weight percent in the mixture (Equation 2). High HLB values indicate relatively hydrophilic polymer matrices, while low HLB values indicate more hydrophobic polymer matrices.

The polyester polymers used were:

Poly(D,L-Lactide)=PLA, each repeat unit is 72 Da. Poly(D,L-lactide-co-glycolide)=PLGA, each repeat unit of lactide is 72 Da, glycolide is 58 Da. Poly(δ-Valerolactone)=PVL, each repeat unit is 100 Da. Poly(ε-Caprolactone)=PCL, each repeat unit is 114 Da. Poly(ethylene glycol) methyl ether-block-poly(D,L lactide)=PLA-PEG, each unit of lactide is 72 Da, each unit of ethylene glycol is 44.05 Da.

For the H-NMR spectroscopy, a portion of each polymer was dissolved in CDCl₃ and transferred into a glass NMR tube for analysis. The polymer Mn was calculated by comparing the chemical shift of an end group peak (—CH₂—OH, 3.65 ppm, for example), to those peaks commonly known for the repeat units in each polyester. For some of the polymers, the manufacturer provided the H-NMR data in the certificate of analysis. Using Davies's definition, each polyester homopolymer (PLGA, PLA, PVL, PCL), contains hydrophilic groups composed of (—C═O—O—) from each repeat unit, plus (—H), and (—OH), on the ends of the polymer. The sum of these is defined as Mnₕ. For PLA-PEG, the PEG polymer is considered hydrophilic; (—CH₂—CH₂—O) for each repeat unit, plus the end group (—OCH₃), or Mn PEG=Mnₕ PEG. For the PLA block, Mnₕ PLA is calculated as the sum of (—C═O—O—) from each repeat unit, plus the end group (—OH). The block copolymer Mnₕ=Mnₕ PEG+Mnₕ PLA.

$$HLB as determined by {Griffin}^{′} s method \text{:} HLB = 20 \times \frac{{Mw}_{h}}{Mw} ; \qquad Equation 1.$$

where Mwₕ is the molecular weight of the hydrophilic molecules, and Mw is the total molecular weight. Note that Mn (and Mnₕ) were used from H-NMR for the polymers as opposed to Mw (and Mwₕ), by an alternative method (such as gel permeation chromatography or viscometry).

$$HLB of polymer mixtures ( matrix ) \text{:} HLB = \frac{\sum {Mn}_{h} {X}_{i}}{\sum {MnX}_{i}} \times 20 ; \qquad Equation 2.$$

where Mnₕ is the sum of the hydrophilic molecules of each individual polymer, Xᵢ is the weight fraction of each polymer, and Mn is the number average molecular weight of each polymer in the matrix. For example, a two polymer matrix (P1 and P2), the HLB is calculated as:

$$\frac{( {Mn}_{h} P 1 \times {X}_{i} P 1 ) + ( {Mn}_{h} P 2 \times {X}_{i} P 2 )}{( Mn P 1 \times {X}_{i} P 1 ) + ( Mn P 2 \times {X}_{i} P 2 )} \times 20$$

Thus, for an exemplary formulation, the formulation consists of 100% poly(D,L-lactide), (PLA). The polymer was purchased from Sigma Aldrich, catalog number 719978-5G, lot number STBD7024V.

From the H-NMR spectrum, the polymer was determined to have 52.8 units of lactide. The HLB was calculated using Equation 1: Mn=52.8 units×72 Da/unit+18 Da (OH+H)=3819.6 Da; Mnₕ=52.8 units (—C═O—O—)×44 Da/unit+18 Da (OH+H)=2341.2 Da;

$$HLB PLA = \frac{2341.2 Da}{3819.6 Da} \times 20 = 12.26 .$$

As another example, the formulation contains a polymer matrix of 75% PLGA (Evonik part number Resomer RG 502 H,) and 25% PLA-PEG (Evonik part number Resomer Select 100 DL mPEG 5000 (15 wt % PEG)).

The HLB for the polymer matrix was calculated using Equation 2. From the H-NMR for PLGA, 90.1 units of lactide and 97.6 units of glycolide were calculated. Mn=(90.1 units lactide×72 Da/unit lactide)+(97.6 units glycolide×58 Da/unit glycolide)+18 Da (OH+H)=12,166.0 Da; Mnₕ=(90.1 units lactide (—C═O—O—)×44 Da/unit)+(97.6 units glycolide (—C═O—O—)×44 Da/unit)+18 Da (OH+H)=8276.8 Da. The Mn for PLA-PEG by H-NMR was reported by Evonik in the certificate of analysis for this polymer (Mn PEG=4753 Da, Mn PLA-PEG=31,470 Da). Mn PEG was given by the manufacturer=4753 Da, therefore Mnₕ PEG=4753.0 Da; Mnₕ PLA=PLA-PEG Mn 31,470.0 Da-4753.0 Da PEG=26,717.0 Da PLA; 26717.0 Da PLA−17 Da (—OH)=26,700.0 Da PLA repeat units; 26,700.0 Da PLA/(72 Da/repeat unit lactide)=370.8 units; Mnₕ PLA=370.8 units (—C═O—O—)×44 Da/unit+17 Da (—OH)=16332.2 Da; Mnₕ PLA-PEG=4753.0 Da Mnₕ PEG+16332.2 Da Mnₕ PLA=21085.2 Da. The HLB of the matrix used in the formulation was then calculated using Equation 2:

$$HLB Matrix = \frac{( 8276.8 Da \times 75 \% + 21085.2 Da \times 25 \% )}{( 12166.0 Da \times 75 \% + 31470.0 Da \times 25 \% )} \times 20 = 13.51$$

Relevant data are shown in the tables below.

TABLE 5
Example characteristics for calculations

| | | | H-NMR | | Mn | Mn | Total | Units | Units | Units | Units | Units |
|---|---|---|---|---|---|---|---|---|---|---|---|---|
| Polymer | Manufacturer | Cat# | Provider | Solvent | Core | PEG | Mn | LA | GA | CL | VL | EG |
| Poly(delta- | Akina/Polyscitech | AP115 | Akina | CDCl₃ | 26508.0 | — | 26508.0 | — | — | — | 264.9 | — |
| Valerolactone) | | | | | | | | | | | | |
| Resomer R 202 H | Sigma Aldrich | 719978-5G | Selecta | CDCl₃ | 3819.6 | — | 3819.6 | 52.8 | — | — | — | — |
| Resomer R 202 H | Sigma Aldrich | 719978-5G | Selecta | CDCl₃ | 5115.6 | — | 5115.6 | 70.8 | — | — | — | — |
| Resomer R 202 H | Evonik | Resomer R | Selecta | CDCl₃ | 4352.4 | — | 4352.4 | 60.2 | — | — | — | — |
| | | 202 H | | | | | | | | | | |
| Polycaprolactone | Sigma Aldrich | 440752-250G | Selecta | CDCl₃ | 4988.4 | — | 4988.4 | — | — | 43.6 | — | — |
| 14k | | | | | | | | | | | | |
| Polycaprolactone | Sigma Aldrich | 440752-250G | Selecta | CDCl₃ | 4806.0 | — | 4806.0 | — | — | 42.0 | — | — |
| 14k | | | | | | | | | | | | |
| Resomer Select | Evonik | 5050 DLG | Selecta | CDCl₃ | 10612.2 | — | 10612.2 | 83.1 | 79.5 | — | — | — |
| 5050 DLG 2.5A | | 2.5A | | | | | | | | | | |
| Resomer RG 502 H | Evonik | RG 502 H | Selecta | CDCl₃ | 12166.0 | — | 12166.0 | 90.1 | 97.6 | — | — | — |
| Resomer RG 502 H | Evonik | RG 502 H | Selecta | CDCl₃ | 10809.2 | — | 10809.2 | 80.6 | 86.0 | — | — | — |
| Resomer Select | Evonik | Resomer | Evonik | CDCl₃ | 26717.0 | 4753.0 | 31470.0 | 370.8 | — | — | — | 107.2 |
| 100 DL mPEG | | Select 100 DL | | | | | | | | | | |
| 5000 (15 wt % | | mPEG 5000 | | | | | | | | | | |
| PEG) | | (15 wt % PEG) | | | | | | | | | | |
| Resomer Select | Evonik | Resomer | Selecta | CDCl₃ | 14107.4 | 4638.6 | 18746.0 | 195.7 | — | — | — | 104.6 |
| 100 DL mPEG | | Select 100 DL | | | | | | | | | | |
| 5000 4CE | | mPEG 5000 | | | | | | | | | | |
| | | 4CE | | | | | | | | | | |
| Resomer Select | Evonik | Resomer | Selecta | CDCl₃ | 5193.8 | 5299.4 | 10493.2 | 71.9 | — | — | — | 119.6 |
| 100 DL mPEG | | Select 100 DL | | | | | | | | | | |
| 5000 (54 wt % | | mPEG 5000 | | | | | | | | | | |
| PEG) | | (54 wt % PEG) | | | | | | | | | | |

TABLE 6
Ovalbumin formulations

| | | | | | | | PLA-PEG, | PLA- | | |
|---|---|---|---|---|---|---|---|---|---|---|
| | Core | | Core | | | PLA-PEG | PLA Units | PEG, PEG | PLA-PEG | Combined |
| Formulation | Polymer | Block Copolymer | Units (#) | Core Mn | Core Mn H | Mn | (#) | Units (#) | Mn H | HLB |
| 1 | PLGA | — | 90.1:97.6 | 12166.0 | 8276.8 | — | — | — | — | 13.61 |
| | | | (LA:GA) | | | | | | | |
| 2 | PLGA | PLA-PEG (15 wt % PEG) | 90.1:97.6 | 12166.0 | 8276.8 | 31470.0 | 370.8 | 107.2 | 21085.2 | 13.51 |
| | | | (LA:GA) | | | | | | | |
| 3 | PLGA | PLA-PEG (25 wt % PEG) | 90.1:97.6 | 12166.0 | 8276.8 | 18746.0 | 195.7 | 104.6 | 13266.4 | 13.79 |
| | | | (LA:GA) | | | | | | | |
| 4 | PLGA | PLA-PEG 5000 (54 wt % | 90.1:97.6 | 12166.0 | 8276.8 | 10493.2 | 71.9 | 119.6 | 8480.0 | 14.18 |
| | | PEG) | (LA:GA) | | | | | | | |
| 5 | PLA | — | 52.8 | 3819.6 | 2341.2 | — | — | — | — | 12.26 |
| 6 | PLA | PLA-PEG (15 wt % PEG) | 70.8 | 5115.6 | 3133.2 | 31470.0 | 370.8 | 107.2 | 21085.2 | 13.02 |
| 7 | PLA | PLA-PEG (25 wt % PEG) | 70.8 | 5115.6 | 3133.2 | 18746.0 | 195.7 | 104.6 | 13266.4 | 13.30 |
| 8 | PLA | PLA-PEG 5000 (54 wt % | 70.8 | 5115.6 | 3133.2 | 10493.2 | 71.9 | 119.6 | 8480.0 | 13.84 |
| | | PEG) | | | | | | | | |
| 9 | PVL | — | 264.9 | 26508.0 | 11673.6 | — | — | — | — | 8.81 |
| 10 | PVL | PLA-PEG (15 wt % PEG) | 264.9 | 26508.0 | 11673.6 | 31470.0 | 370.8 | 107.2 | 21085.2 | 10.11 |
| 11 | PVL | PLA-PEG (25 wt % PEG) | 264.9 | 26508.0 | 11673.6 | 18746.0 | 195.7 | 104.6 | 13266.4 | 9.83 |
| 12 | PVL | PLA-PEG 5000 (54 wt % | 264.9 | 26508.0 | 11673.6 | 10493.2 | 71.9 | 119.6 | 8480.0 | 9.66 |
| | | PEG) | | | | | | | | |
| 13 | PCL | — | 42.0 | 4806.0 | 1866.0 | — | — | — | — | 7.77 |
| 14 | PCL | PLA-PEG (15 wt % PEG) | 42.0 | 4806.0 | 1866.0 | 31470.0 | 370.8 | 107.2 | 21085.2 | 11.63 |
| 15 | PCL | PLA-PEG (25 wt % PEG) | 42.0 | 4806.0 | 1866.0 | 18746.0 | 195.7 | 104.6 | 13266.4 | 11.38 |
| 16 | PCL | PLA-PEG 5000 (54 wt % | 42.0 | 4806.0 | 1866.0 | 10493.2 | 71.9 | 119.6 | 8480.0 | 11.30 |
| | | PEG) | | | | | | | | |
| 17 | PLGA | PLA-PEG (15 wt % PEG) | 80.6:86.0 | 10809.2 | 7348.4 | 31470.0 | 370.8 | 107.2 | 21085.2 | 13.50 |
| | | | (LA:GA) | | | | | | | |
| 18 | PLGA | PLA-PEG (25 wt % PEG) | 83.1:79.5 | 10612.2 | 7172.4 | 18746.0 | 195.7 | 104.6 | 13266.4 | 13.75 |
| | | | (LA:GA) | | | | | | | |

TABLE 7
CPE Formulations (19-32)

| | | | | | | PLA-PEG, | PLA-PEG, | | |
|---|---|---|---|---|---|---|---|---|---|
| Core | | Core | | | PLA-PEG | PLA Units | PEG Units | PLA-PEG | Calculated |
| Polymer | Block Copolymer | Units (#) | Core Mn | Core Mn H | Mn | (#) | (#) | Mn H | HLB |
| PLGA | — | 83.1:79.5 | 10612.2 | 7172.4 | — | — | — | — | 13.52 |
| | | (LA:GA) | | | | | | | |
| PLGA | PLA-PEG (15 wt % PEG) | 83.1:79.5 | 10612.2 | 7172.4 | 31470.0 | 370.8 | 107.2 | 21085.2 | 13.46 |
| | | (LA:GA) | | | | | | | |
| PLGA | PLA-PEG (25 wt % PEG) | 83.1:79.5 | 10612.2 | 7172.4 | 18746.0 | 195.7 | 104.6 | 13266.4 | 13.75 |
| | | (LA:GA) | | | | | | | |
| PLGA | PLA-PEG 5000 (54 wt % | 83.1:79.5 | 10612.2 | 7172.4 | 10493.2 | 71.9 | 119.6 | 8480.0 | 14.17 |
| | PEG) | (LA:GA) | | | | | | | |
| PLA | — | 52.8 | 3819.6 | 2341.2 | — | — | — | — | 12.26 |
| PLA | PLA-PEG (15 wt % PEG) | 52.8 | 3819.6 | 2341.2 | 31470.0 | 370.8 | 107.2 | 21085.2 | 13.10 |
| PLA | PLA-PEG (25 wt % PEG) | 70.8 | 5115.6 | 3133.2 | 18746.0 | 195.7 | 104.6 | 13266.4 | 13.30 |
| PLA | PLA-PEG 5000 (54 wt % | 70.8 | 5115.6 | 3133.2 | 10493.2 | 71.9 | 119.6 | 8480.0 | 13.84 |
| | PEG) | | | | | | | | |
| PCL | — | 43.6 | 4970.4 | 1936.4 | — | — | — | — | 7.79 |
| PCL | PLA-PEG (15 wt % PEG) | 42.0 | 4806.0 | 1866.0 | 31470.0 | 370.8 | 107.2 | 21085.2 | 11.63 |
| PCL | PLA-PEG (25 wt % PEG) | 42.0 | 4806.0 | 1866.0 | 18746.0 | 195.7 | 104.6 | 13266.4 | 11.38 |
| PCL | PLA-PEG 5000 (54 wt % | 42.0 | 4806.0 | 1866.0 | 10493.2 | 71.9 | 119.6 | 8480.0 | 11.30 |
| | PEG) | | | | | | | | |
| PLGA | PLA-PEG (15 wt % PEG) | 83.1:79.5 | 10612.2 | 7172.4 | 31470.0 | 370.8 | 107.2 | 21085.2 | 13.46 |
| | | (LA:GA) | | | | | | | |
| PLGA | PLA-PEG (25 wt % PEG) | 83.1:79.5 | 10612.2 | 7172.4 | 18746.0 | 195.7 | 104.6 | 13266.4 | 13.75 |
| | | (LA:GA) | | | | | | | |

### Example 5: Relationship Between Polypeptide Allergen Added to Formulation and Surface Recognition by ELISA

An ELISA was used with the formed nanocarriers (produced as described above) to observe binding of allergen specific IgG antibodies to the nanocarrier surfaces as a model for IgE interactions in vivo. Nanocarriers were prepared containing ovalbumin (OVA), with various volume and concentration of the allergen and 100 mg of polymer matrix. Control formulations of polymer matrix only or very high amounts of added allergen were also produced to act as controls. It was observed that the more allergen that was added, the more surface recognition by IgG antibodies.

Next, various formulations were prepared. The results indicate that lots with very low HLB exhibited low encapsulation/load. Such lots can be seen generally with the PCL and PVL only formulations. Unexpectedly, it was discovered that there is a region within a polymer matrix relative hydrophobicity space which is optimal for allergen load and the minimization of antibody binding of polypeptides displayed on the surface of nanocarriers. When this is used to prepare nanocarriers containing allergen, such as ovalbumin, the probability of hypersensitivity reactions in sensitive individuals receiving treatment is expected to be minimized, potentially improving the safety of the therapy.

TABLE 8
Effect of nanocarrier polymer matrix on allergen surface recognition by ELISA
Ovalbumin Formulations

| Formulation | | | | | Ova Load | | Ova ELISA |
|---|---|---|---|---|---|---|---|
| Reference | Core Polymer | Block Copolymer | % PEG | Size (nm) | (%) | Calc. HLB | Top OD |
| 1 | PLGA | — | 0 | 122 | 0.62 | 13.61 | 0.566 |
| 2 | PLGA | PLA-PEG (15 wt % PEG) | 3.75 | 118 | 0.94 | 13.51 | 0.750 |
| 3 | PLGA | PLA-PEG (25 wt % PEG) | 6.25 | 112 | 0.87 | 13.79 | 0.885 |
| 4 | PLGA | PLA-PEG 5000 (54 wt % PEG) | 13.5 | 91 | 0.97 | 14.18 | 0.981 |
| 5 | PLA | — | 0 | 123 | 0.75 | 12.26 | 0.533 |
| 6 | PLA | PLA-PEG (15 wt % PEG) | 3.75 | 134 | 1.28 | 13.02 | 0.588 |
| 7 | PLA | PLA-PEG (25 wt % PEG) | 6.25 | 128 | 0.84 | 13.30 | 0.586 |
| 8 | PLA | PLA-PEG 5000 (54 wt % PEG) | 13.5 | 113 | 0.87 | 13.84 | 0.655 |
| 9 | PVL | — | 0 | 170 | 1.42 | 8.81 | 0.874 |
| 10 | PVL | PLA-PEG (15 wt % PEG) | 3.75 | 163 | 0.84 | 10.11 | 0.965 |
| 11 | PVL | PLA-PEG (25 wt % PEG) | 6.25 | 155 | 0.81 | 9.83 | 0.867 |
| 12 | PVL | PLA-PEG 5000 (54 wt % PEG) | 13.5 | 145 | 0.65 | 9.66 | 0.827 |
| 13 | PCL | — | 0 | 163 | 0.09 | 7.77 | 0.735 |
| 14 | PCL | PLA-PEG (15 wt % PEG) | 3.75 | 156 | 0.72 | 11.63 | 1.099 |
| 15 | PCL | PLA-PEG (25 wt % PEG) | 6.25 | 155 | 0.64 | 11.38 | 1.064 |
| 16 | PCL | PLA-PEG 5000 (54 wt % PEG) | 13.5 | 149 | 0.22 | 11.30 | 1.042 |
| 17 | PLGA | PLA-PEG (15 wt % PEG) | 3.75 | 155 | 7.50 | 13.50 | 2.096 |
| 18 | PLGA | PLA-PEG (25 wt % PEG) | 6.25 | 155 | 0.00 | 13.75 | 0.014 |

### Example 6: Relationship Between Polypeptide Allergen Added to Formulation and Surface Recognition by ELISA

An ELISA was used with the formulations (as described above) to measure binding of the CPE specific IgG antibodies to the nanocarrier surfaces. Control formulations of polymer matrix only or relatively high allergen load were also produced to act as controls for an ELISA assay. Again, the more allergen that was added, the more surface recognition.

Next, various formulations were prepared using CPE allergen with different polymer matrices. Each formulation was evaluated for both CPE and Ara h2 specific surface ELISA as one of the major allergens in the CPE mixture. Polymer matrices that are too hydrophilic, result in higher surface detectable allergen. On the other side of the HLB scale, formulations such as with PCL as the core polymer, result in either near total loss of the allergen from the formulation or ELISA values that are substantially higher (such as compared to those prepared with PLA or PLGA). There appears to be a lower limit on the HLB scale of 11 according to this example.

TABLE 9
Effect of nanocarrier polymer matrix on CPE surface recognition by ELISA
CPE Formulations

| | | | | | | | | Arah2 |
|---|---|---|---|---|---|---|---|---|
| Lot | Core | | | Size | CPE Load | Calculated | CPE IgG1 Top | IgG Top |
| Number | Polymer | Block Copolymer | PEG (%) | (nm) | (%) | HLB | OD | OD |
| 19 | PLGA | — | 0 | 138 | 0.95 | 13.52 | 1.496 | 0.370 |
| 20 | PLGA | PLA-PEG (15 wt % PEG) | 3.75 | 121 | 1.15 | 13.46 | 1.608 | 0.332 |
| 21 | PLGA | PLA-PEG (25 wt % PEG) | 6.25 | 118 | 1.19 | 13.75 | 1.659 | 0.508 |
| 22 | PLGA | PLA-PEG 5000 (54 wt % PEG) | 13.5 | 109 | 1.19 | 14.17 | 1.725 | 0.484 |
| 23 | PLA | — | 0 | 124 | 1.17 | 12.26 | 0.978 | 0.246 |
| 24 | PLA | PLA-PEG (15 wt % PEG) | 3.75 | 127 | 1.38 | 13.10 | 1.601 | 0.457 |
| 25 | PLA | PLA-PEG (25 wt % PEG) | 6.25 | 122 | 1.13 | 13.30 | 2.046 | 0.554 |
| 26 | PLA | PLA-PEG 5000 (54 wt % PEG) | 13.5 | 99 | 0.74 | 13.84 | 1.834 | 0.322 |
| 27 | PCL | — | 0 | 151 | 0.50 | 7.76 | 0.508 | 0.120 |
| 28 | PCL | PLA-PEG (15 wt % PEG) | 3.75 | 153 | 0.84 | 11.63 | 2.214 | 1.005 |
| 29 | PCL | PLA-PEG (25 wt % PEG) | 6.25 | 147 | 0.78 | 11.38 | 2.214 | 0.988 |
| 30 | PCL | PLA-PEG 5000 (54 wt % PEG) | 13.5 | 144 | 0.60 | 11.30 | 2.244 | 0.995 |
| 31 | PLGA | PLA-PEG (15 wt % PEG) | 3.75 | 140 | 0.93 | 13.46 | 2.337 | 1.640 |
| 32 | PLGA | PLA-PEG (25 wt % PEG) | 6.25 | 155 | 0.00 | 13.75 | 0.021 | 0.018 |

### Example 7: Relationship Between Wheat Allergen Added to Nanocarrier Formulations and Surface Recognition by ELISA

Initial nanocarrier formulations were prepared containing raw wheat gliadin using the double emulsion method, modifying the allergen concentration and volume, keeping other composition and processing parameters constant. BALB/c mice were inoculated with the same RW gliadin adsorbed to alum to generate the antibody serum for the ELISA. Nanocarrier formulations were then used to coat the ELISA plates for surface RW gliadin determination.

If the amount of RW gliadin added to the formulation is divided by the surface area of the formed nanocarriers, a relationship with the surface ELISA data can be seen. It was observed that increasing the RW gliadin added to the formulation results in higher surface ELISA OD values, which is consistent with other ovalbumin, house dust mite, ragweed, and peanut extract allergen data. To discriminate between the various polymer matrices, other formulations using a 2% target load for raw wheat gliadin were explored.

### Example 8: Relationship Between Polymer Matrix Relative Hydrophobicity and Surface ELISA Data

Various nanocarrier formulations were prepared using a 2% target load for raw wheat gliadin with different polymer compositions (matrix). Two polymer matrix parameters were explored, core polymer and weight % PEG. For the surface ELISA, a polymer only formulation without added allergen was used as the negative control, and the highest surface ELISA measured as the positive control.

TABLE 10
Raw wheat gliadin formulations modifying the allergen aqueous phase

| | | | | | | | | RW |
|---|---|---|---|---|---|---|---|---|
| | | | RW- | Surface | RW-Gliadin | | | Gliadin |
| | PEG | Size | Gliadin | Area | (mg)/NP SA | Load | Calculated | Top |
| Block Copolymer | (wt %) | (nm) | (mg) | (nm²) | (nm²) | (%) | HLB | OD |
| PLA-PEG (15 wt % | 3.75 | 115 | 5.0 | 4.1.E+04 | 1.2.E−04 | 1.05 | 13.46 | 0.668 |
| PEG) | | | | | | | | |
| PLA-PEG (15 wt % | 3.75 | 124 | 2.0 | 4.8.E+04 | 4.2.E−05 | 0.86 | 13.46 | 0.530 |
| PEG) | | | | | | | | |
| PLA-PEG (15 wt % | 3.75 | 117 | 1.5 | 4.3.E+04 | 3.5.E−05 | 0.81 | 13.46 | 0.560 |
| PEG) | | | | | | | | |
| PLA-PEG (15 wt % | 3.75 | 119 | 1.0 | 4.4.E+04 | 2.3.E−05 | 0.72 | 13.46 | 0.557 |
| PEG) | | | | | | | | |
| PLA-PEG (15 wt % | 3.75 | 118 | 1.0 | 4.4.E+04 | 2.3.E−05 | 0.37 | 13.46 | 0.470 |
| PEG) | | | | | | | | |
| PLA-PEG (15 wt % | 3.75 | 123 | 1.0 | 4.7.E+04 | 2.1.E−05 | 0.58 | 13.46 | 0.528 |
| PEG) | | | | | | | | |
| PLA-PEG (15 wt % | 3.75 | 123 | 1.0 | 4.7.E+04 | 2.1.E−05 | 0.85 | 13.46 | 0.505 |
| PEG) | | | | | | | | |
| PLA-PEG (15 wt % | 3.75 | 125 | 1.0 | 4.9.E+04 | 2.1.E−05 | 0.81 | 13.46 | 0.463 |
| PEG) | | | | | | | | |

TABLE 11
Effect of nanocarrier polymer matrix HLB on RW gliadin surface ELISA

| | | | | | | RW |
|---|---|---|---|---|---|---|
| | | | | | Calculated | Gliadin |
| Core | Block Copolymer | PEG (wt %) | Size (nm) | Load (%) | HLB | Top OD |
| PLGA | — | 0 | 119 | 1.00 | 13.52 | 0.141 |
| PLGA | PLA-PEG (15 wt % PEG) | 3.75 | 118 | 0.60 | 13.44 | 0.181 |
| PLGA | PLA-PEG (25 wt % PEG) | 6.25 | 122 | 0.56 | 13.72 | 0.158 |
| PLGA | PLA-PEG (54 wt % PEG) | 13.5 | 103 | 0.30 | 14.35 | 0.157 |
| PLA | — | 0 | 134 | 1.08 | 12.22 | 0.187 |
| PLA | PLA-PEG (15 wt % PEG) | 3.75 | 134 | 1.23 | 12.36 | 0.183 |
| PLA | PLA-PEG (25 wt % PEG) | 6.25 | 132 | 1.08 | 13.08 | 0.177 |
| PLA | PLA-PEG (54 wt % PEG) | 13.5 | 135 | 0.93 | 13.61 | 0.141 |
| PCL | — | 0 | 138 | 0.41 | 7.71 | 0.140 |
| PCL | PLA-PEG (15 wt % PEG) | 3.75 | 144 | 0.29 | 10.44 | 0.236 |
| PCL | PLA-PEG (25 wt % PEG) | 6.25 | 150 | 0.45 | 10.29 | 0.377 |
| PCL | PLA-PEG (54 wt % PEG) | 13.5 | 137 | 0.41 | 10.03 | 0.230 |
| PLGA | PLA-PEG (15 wt % PEG) | 6.25 | 155 | — | — | 0.069 |
| PLGA | PLA-PEG (15 wt % PEG) | 3.75 | 131 | 1.05 | 13.46 | 0.283 |

From the data, PCL core polymer (with or without PLA-PEG copolymer), results in substantial loss of raw wheat gliadin, and/or higher surface ELISA OD values compared to PLA and PLGA formulations. Given the PCL data, there appears to be an optimized HLB limit range, for this example, of between 10.44 and 12.22. Formulations with PLA display similar low surface ELISA OD values and allergen loads around 1%. For PLGA as the core polymer, there is a substantial drop in RW gliadin load between 6.25% and 13.5% PEG weight percent, representing an optimized upper HLB limit of 13.72, for this example.

### Example 9: Relationship Between Polypeptide Allergen Added to the Formulation and Surface Recognition by ELISA

Initial formulations were prepared using a double emulsion method, containing house dust mite extract (HDM) or Ragweed extract (Ragweed). The formulations modified the allergen aqueous phase volume and allergen concentration, keeping other composition and processing parameters constant.

TABLE 12
HDM formulations modifying the allergen aqueous phase

| | | | | | | | HDM | | | |
|---|---|---|---|---|---|---|---|---|---|---|
| | | | | | | Surface | (mg)/NP | | | HDM |
| | | Block | PEG | Size | HDM | Area | SA | Load | Calculated | Top |
| Comments | Core | Copolymer | (wt %) | (nm) | (mg) | (nm²) | (nm²) | (%) | HLB | OD |
| W1[0.5 mL | PLGA | PLA-PEG (15 wt | 3.75 | 131 | 5.0 | 5.4.E+04 | 9.3.E−05 | 4.14 | 13.46 | 1.920 |
| 10 mg/mL] | | % PEG) | | | | | | | | |
| W1[0.2 mL | PLGA | PLA-PEG (15 wt | 3.75 | 120 | 2.0 | 4.5.E+04 | 4.4.E−05 | 2.04 | 13.46 | 1.462 |
| 10 mg/mL] | | % PEG) | | | | | | | | |
| W1[0.15 mL | PLGA | PLA-PEG (15 wt | 3.75 | 122 | 1.5 | 4.7.E+04 | 3.2.E−05 | 1.62 | 13.46 | 1.200 |
| 10 mg/mL] | | % PEG) | | | | | | | | |
| W1[0.1 mL | PLGA | PLA-PEG (15 wt | 3.75 | 107 | 1.0 | 3.6.E+04 | 2.8.E−05 | 1.03 | 13.46 | 1.049 |
| 10 mg/mL] | | % PEG) | | | | | | | | |
| W1[0.5 mL | PLGA | PLA-PEG (15 wt | 3.75 | 128 | 1.0 | 5.1.E+04 | 1.9.E−05 | 1.15 | 13.46 | 0.683 |
| 2 mg/mL] | | % PEG) | | | | | | | | |
| W1[0.2 mL | PLGA | PLA-PEG (15 wt | 3.75 | 114 | 1.0 | 4.1.E+04 | 2.4.E−05 | 1.17 | 13.46 | 0.974 |
| 5 mg/mL] | | % PEG) | | | | | | | | |
| W1[0.15 mL | PLGA | PLA-PEG (15 wt | 3.75 | 112 | 1.0 | 3.9.E+04 | 2.6.E−05 | 0.95 | 13.46 | 1.066 |
| 6.7 mg/mL] | | % PEG) | | | | | | | | |
| W1[0.05 mL | PLGA | PLA-PEG (15 wt | 3.75 | 106 | 1.0 | 3.5.E+04 | 2.8.E−05 | 0.38 | 13.46 | 0.893 |
| 20 mg/mL] | | % PEG) | | | | | | | | |

TABLE 13
Ragweed formulations modifying the allergen aqueous phase

| | | | | | | | Ragweed | | | |
|---|---|---|---|---|---|---|---|---|---|---|
| | | Block | PEG | Size | Ragweeed | Surface | (mg)/NP | Load | Calculated | Ragweed |
| Comments | Core | Copolymer | (wt %) | (nm) | (mg) | Area (nm²) | SA (nm²) | (%) | HLB | Top OD |
| W1[0.5 mL | PLGA | PLA-PEG | 3.75 | 134 | 5.0 | 5.6.E+04 | 8.9.E−05 | 4.55 | 13.46 | 1.299 |
| 10 mg/mL] | | (15 wt % | | | | | | | | |
| | | PEG) | | | | | | | | |
| W1[0.2 mL | PLGA | PLA-PEG | 3.75 | 120 | 2.0 | 4.5.E+04 | 4.4.E−05 | 2.22 | 13.46 | 0.815 |
| 10 mg/mL] | | (15 wt % | | | | | | | | |
| | | PEG) | | | | | | | | |
| W1[0.15 mL | PLGA | PLA-PEG | 3.75 | 118 | 1.5 | 4.4.E+04 | 3.4.E−05 | 1.64 | 13.46 | 0.535 |
| 10 mg/mL] | | (15 wt % | | | | | | | | |
| | | PEG) | | | | | | | | |
| W1[0.1 mL | PLGA | PLA-PEG | 3.75 | 118 | 1.0 | 4.4.E+04 | 2.3.E−05 | 1.14 | 13.46 | 0.469 |
| 10 mg/mL] | | (15 wt % | | | | | | | | |
| | | PEG) | | | | | | | | |
| W1[0.5 mL | PLGA | PLA-PEG | 3.75 | 122 | 1.0 | 4.7.E+04 | 2.1.E−05 | 1.20 | 13.46 | 0.332 |
| 2 mg/mL] | | (15 wt % | | | | | | | | |
| | | PEG) | | | | | | | | |
| W1[0.2 mL | PLGA | PLA-PEG | 3.75 | 116 | 1.0 | 4.2.E+04 | 2.4.E−05 | 1.18 | 13.46 | 0.385 |
| 5 mg/mL] | | (15 wt % | | | | | | | | |
| | | PEG) | | | | | | | | |
| W1[0.15 mL | PLGA | PLA-PEG | 3.75 | 113 | 1.0 | 4.0.E+04 | 2.5.E−05 | 1.34 | 13.46 | 0.476 |
| 6.7 mg/mL] | | (15 wt % | | | | | | | | |
| | | PEG) | | | | | | | | |
| W1[0.05 mL | PLGA | PLA-PEG | 3.75 | 108 | 1.0 | 3.7.E+04 | 2.7.E−05 | 0.56 | 13.46 | 0.599 |
| 20 mg/mL] | | (15 wt % | | | | | | | | |
| | | PEG) | | | | | | | | |

The W1 phase is the aqueous allergen solution which is homogenized into the polymer organic phase (primary emulsion). To measure the surface antibody binding to the nanocarriers by ELISA, BALB/c mice were inoculated with the indicated allergen adsorbed to alum to generate allergen specific antibody serum. Nanocarrier formulations were then coated onto 96-well ELISA plates and screened using the respective allergen mouse serum. A linear relationship with the amount of antibody binding to the surface is seen when the amount of allergen added to the formulation is divided by the surface area of the formed synthetic nanocarriers.

Thus, the more allergen is added, the higher the surface ELISA OD values, which is consistent with other ovalbumin, wheat extract, and peanut extract allergen data. To minimize surface allergen and to discriminate between the various polymer matrices, other formulations using a 1% target load for HDM and ragweed were explored.

### Example 10. Relationship Between Polymer Composition and Surface Recognition by ELISA

Various formulations were prepared using 1% target load for ragweed and dust mite extracts with different polymer mixtures. Two parameters were explored, core polymer and weight % PEG. For the surface ELISA, a polymer only formulation without added allergen was used as the negative control, and the highest surface ELISA measured for each allergen as the positive control.

TABLE 14
Effect of nanocarrier polymer matrix HLB on HDM surface ELISA

| | | | | | Calculated | HDM Top |
|---|---|---|---|---|---|---|
| Core | Block Copolymer | PEG (wt %) | Size (nm) | Load (%) | HLB | OD |
| PLGA | — | 0 | 113 | 0.55 | 13.52 | 0.925 |
| PLGA | PLA-PEG (15 wt % PEG) | 3.75 | 111 | 0.73 | 13.44 | 1.010 |
| PLGA | PLA-PEG (25 wt % PEG) | 6.25 | 104 | 0.55 | 13.72 | 1.101 |
| PLGA | PLA-PEG (54 wt % PEG) | 13.5 | 95 | 0.57 | 14.35 | 1.196 |
| PLA | — | 0 | 127 | 0.58 | 12.22 | 0.955 |
| PLA | PLA-PEG (15 wt % PEG) | 3.75 | 117 | 0.60 | 12.36 | 1.033 |
| PLA | PLA-PEG (25 wt % PEG) | 6.25 | 118 | 0.78 | 13.08 | 0.985 |
| PLA | PLA-PEG (54 wt % PEG) | 13.5 | 114 | 0.56 | 13.61 | 0.971 |
| PCL | — | 0 | 153 | 0.00 | 7.71 | 0.759 |
| PCL | PLA-PEG (15 wt % PEG) | 3.75 | 154 | 0.06 | 10.44 | 1.353 |
| PCL | PLA-PEG (25 wt % PEG) | 6.25 | 155 | 0.05 | 10.29 | 1.317 |
| PCL | PLA-PEG (54 wt % PEG) | 13.5 | 145 | 0.01 | 10.03 | 1.051 |
| PLGA | PLA-PEG (15 wt % PEG) | 6.25 | 155 | — | — | 0.035 |
| PLGA | PLA-PEG (15 wt % PEG) | 3.75 | 131 | 4.14 | 13.46 | 1.920 |

TABLE 15
Effect of nanocarrier polymer matrix HLB on ragweed surface ELISA

| | | | | | Calculated | Ragweed |
|---|---|---|---|---|---|---|
| Core | Block Copolymer | PEG (wt %) | Size (nm) | Load (%) | HLB | Top OD |
| PLGA | — | 0 | 108 | 0.67 | 13.52 | 0.249 |
| PLGA | PLA-PEG (15 wt % PEG) | 3.75 | 109 | 0.84 | 13.44 | 0.398 |
| PLGA | PLA-PEG (25 wt % PEG) | 6.25 | 111 | 0.89 | 13.72 | 0.402 |
| PLGA | PLA-PEG (54 wt % PEG) | 13.5 | 106 | 0.91 | 14.35 | 0.467 |
| PLA | — | 0 | 123 | 0.61 | 12.22 | 0.348 |
| PLA | PLA-PEG (15 wt % PEG) | 3.75 | 128 | 0.79 | 12.36 | 0.419 |
| PLA | PLA-PEG (25 wt % PEG) | 6.25 | 127 | 0.79 | 13.08 | 0.334 |
| PLA | PLA-PEG (54 wt % PEG) | 13.5 | 112 | 0.65 | 13.61 | 0.399 |
| PCL | — | 0 | 151 | 0.00 | 7.71 | 0.196 |
| PCL | PLA-PEG (15 wt % PEG) | 3.75 | 149 | 0.10 | 10.44 | 0.637 |
| PCL | PLA-PEG (25 wt % PEG) | 6.25 | 152 | 0.08 | 10.29 | 0.778 |
| PCL | PLA-PEG (54 wt % PEG) | 13.5 | 146 | 0.02 | 10.03 | 0.475 |
| PLGA | PLA-PEG (15 wt % PEG) | 6.25 | 155 | — | — | 0.020 |
| PLGA | PLA-PEG (15 wt % PEG) | 3.75 | 134 | 4.55 | 13.46 | 1.299 |

From the data, and consistent with other allergen data, PCL core polymer (with or without PLA-PEG copolymer), results in a near total loss of either HDM or ragweed allergens. Given the lower calculated HLB of PCL compared to PLA or PLGA, there is an optimized lower HLB limit between 10.44 and 12.22, for this example. Formulations with PLA have similar allergen loads as PLGA, and display similar surface ELISA OD values regardless of PEG wt %. For PLGA as the core polymer with HDM, there is a small increase in surface ELISA OD from 0 to 13.5 wt % PEG. With ragweed there is a substantial increase in surface ELISA from 0 to 3.75 PEG wt %, followed by a gradual increase in surface ELISA to 13.5 PEG wt %, although this increase is still lower than the positive control. Thus, for HDM and ragweed, an optimized HLB range is 12.22 to 14.35, for this example.

### Example 11. Analysis of Allergen Solutions by RP-UHPLC

### Materials

Complete peanut extract was provided by Sanofi (82, Avenue Raspail, Gentilly, France), product lot number PS 1140223.

Raw wheat gliadin extract (lyophilized) was prepared as described.

Ovalbumin (lyophilized), was purchased from Worthington Biochemical Corporation (730 Vassar Ave. Lakewood, N.J. 08701), product code LS003054.

Short ragweed, ambrosia artemisiifolia freeze dried extract from Stallergenes Greer (40 Bernard Street—3rd Floor London WC1N 1LE, United Kingdom), product code XP56D3A25.

House Dust Mite, dermatophagoides pteronyssinus freeze dried extract from Stallergenes Greer (40 Bernard Street—3rd Floor London WC1N 1LE, United Kingdom), product code XPB 82D3A25

Phosphate-Buffered Saline (PBS) 1×: solution without Calcium and Magnesium was purchased from Corning Inc. (One Riverfront Plaza Corning, N.Y. 14831), part number 21-040.

### Methods

Allergen solutions were prepared as follows:

Raw wheat gliadin extract solution: 33.58 mg of lyophilized raw wheat gliadin was dissolved in an aqueous solution of 1.70 g 50 mM acetic acid.

Ovalbumin solution: 8.07 mg of lyophilized ovalbumin was dissolved in 0.810 mL of Cellgro PBS 1×.

Short ragweed extract solution: To a vial of lyophilized short ragweed from Greer containing 72.93 mg protein/vial according to the certificate of analysis, 7.293 mL of Cellgro PBS 1× was added.

House dust mite extract solution: To a vial of lyophilized house dust mite from Greer containing 33.45 mg protein/vial according to the certificate of analysis, 3.345 mL of Cellgro PBS 1× was added.

Complete peanut extract solution: 0.104 mL of a stock solution of complete peanut extract containing 48.2 mg/mL protein was diluted in 0.396 mL of Cellgro PBS 1×.

### Analysis of Allergen Solutions by RP-UHPLC:

RP-HPLC was carried out using an Agilent ultra high pressure liquid chromatography instrument (UHPLC). The UHPLC instrument was purchased from Agilent (Agilent Technologies, Santa Clara Calif., USA). The UHPLC was composed of a 1290 Infiniti II DADFS part number G7117A, 1290 MCT part number G7116B, 1290 Vialsampler part number G7129B, and 1290 High Speed Pump part number G7120A. The UHPLC was operated using Agilent OpenLAB CDS ChemStation Edition software, revision C.01.07 SR1. An XBridge Peptide BEH C18 UHPLC column was used, purchased from Waters Corporation (Milford Mass., USA), part number 186003612.

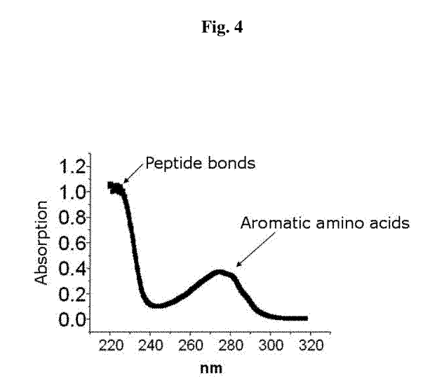

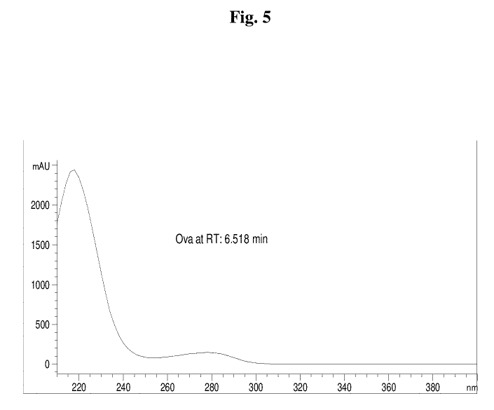

Allergen solutions were monitored by RP-UHPLC at 200 nm absorbance. To distinguish protein from non-protein, the elution profile of each sample was compared to a baseline injection of 20% acetonitrile with a buffer blank injection (in which the protein samples were prepared in). For each observed peak in the chromatogram, the UV/vis absorption spectrum of the peak apex was used to differentiate whether the material is protein related or not compared to a typical protein spectrum (FIG. 4), ovalbumin (FIG. 5), and verses a 0.1% TFA in acetonitrile only reference spectrum. Non-protein profiles were identified and excluded if the chromatographic signal was higher than average, where the known protein load on the column was relatively low, and where absorbance above 260 nm was observed. For each allergen solution, protein peaks were identified and the area under the curve was calculated using the Agilent chromatography software. The weight percent of each peak was then calculated based on the area under the curve divided by the sum of the area under the peaks for all identified protein peaks. The weighted mean retention time for the allergen solution was then calculated using Equation 3. This value can be considered as a metric of relative hydrophobicity since it is directly related to the hydrophobic interaction of the allergen solution with the reverse phase column packing material. For each allergen solution, 3 μg of protein was injected onto the column.

Mobile phase A was composed of 94.9% water, 5% acetonitrile, 0.1% trifluoroacetic acid. Mobile phase B was composed of 19.9% water, 80% acetonitrile, 0.1% trifluoroacetic acid. Acetonitrile was purchased from EMD Millipore part number AX0145-1, and trifluoroacetic acid was purchased from Alfa Aesar part number 44630. Water was supplied by a reverse osmosis deionized water system which was also 0.2 μm filtered.

TABLE 16
Gradient program for each analysis

| Time | % Mobile Phase B | Flow Rate (mL/Minute) |
|---|---|---|
| 0.00 | 0.0 | 3.0 |
| 0.50 | 0.0 | 3.0 |
| 9.50 | 100.0 | 3.0 |
| 10.0 | 0.0 | 3.0 |

Equation 3. Weighted mean retention time (a measure of allergen hydrophobicity) was calculated using the following equation:

$$\overline{x} = \frac{{W}_{1} {X}_{1} + {W}_{2} {X}_{2} … WnXn}{{W}_{1} + {W}_{2} … Wn} ;$$

where W is the weight percent of the total area under the curves for each protein peak in the UHPLC chromatogram. X is the individual protein peak retention times in the UHPLC chromatogram.

The five allergen solutions were analyzed by UHPLC according to the method above. Data for the protein peaks from the chromatograms are below in Table 17 and weighted mean in Table 18.

TABLE 17
Integration of protein peaks from the UHPLC chromatograms

| | | Retention | |
|---|---|---|---|
| Allergen | Peak# | Time (min.) | Area |
| Complete Peanut Extract | 1 | 0.377 | 8% |
| | 2 | 1.978 | 10% |
| | 3 | 2.573 | 4% |
| | 4 | 3.521 | 25% |
| | 5 | 5.022 | 53% |
| Ovalbumin | 1 | 4.872 | 13% |
| | 2 | 6.509 | 87% |
| Raw Wheat Gliadin Extract | 1 | 4.491 | 15% |
| | 2 | 5.137 | 42% |
| | 3 | 5.639 | 24% |
| | 4 | 6.003 | 11% |
| | 5 | 6.514 | 7% |
| Short Ragweed Extract | 1 | 1.569 | 32% |
| | 2 | 3.756 | 4% |
| | 3 | 4.612 | 13% |
| | 4 | 5.011 | 24% |
| | 5 | 5.586 | 20% |
| | 6 | 6.514 | 7% |
| House Dust Mite Extract | 1 | 1.633 | 18% |
| | 2 | 3.363 | 18% |
| | 3 | 3.715 | 9% |
| | 4 | 4.302 | 5% |
| | 5 | 5.203 | 50% |

TABLE 18
Results of weighted average calculations

| Allergen | Result (min.) |
|---|---|
| Complete Peanut Extract | 3.880 |
| Short Ragweed Extract | 4.033 |
| House Dust Mite Extract | 4.037 |
| Raw Wheat Gliadin Extract | 5.356 |
| Ovalbumin | 6.300 |

According to these examples, desirable values for the weighted mean retention time can be between 1 and 10 minutes.

### REFERENCES

1. C Esser and A Radbruch. Immunoglobulin Class Switching: Molecular and Cellular Analysis. Annual Review of Immunology Vol. 8: 717-735 (Volume publication date April 1990) DOI: 10.1146/annurev.iy.08.040190.003441
2. Rudolf Valenta. The future of antigen-specific immunotherapy of allergy. Nature Reviews Immunology 2, 446-453 (June 2002) | doi:10.1038/nri824.
3. Griffin, W. C. Classification of Surface Active Agents by HLB. J. Soc. Cosmet. Chem. 1949, 1, 311-326.
4. Griffin, W. C. Calculation of HLB values of Nonionic Surfactants, J. Soc. Cosmet. Chem. 1954, 5, 249-256.
5. Carlos E. Astete and Cristina M. Sabliov. Synthesis and characterization of PLGA nanoparticles J. Biomater. Sci. Polymer Edn, Vol. 17, No. 3, pp. 247-289 (2006).
6. Petr O. Ilyinskii et al. Adjuvant-carrying synthetic vaccine particles augment the immune response to encapsulated antigen and exhibit strong local immune activation without inducing systemic cytokine release. Vaccine Volume 32, Issue 24, 19 May 2014, Pages 2882-2895.
7. Jean-Christoph Caubet, MD and Julie Wang, MD. Current Understanding of egg allergy. Pediatr Clin North Am. 2011 Apr. 1, 58(2) 427-443,
8. Davies, J. T. A quantitative kinetic theory of emulsion type, I. Physical chemistry of the emulsifying agent. Gas/Liquid and Liquid/Liquid Interfaces. Proceedings of 2nd International Congress Surface Activity, Butterworths, London 1957.
9. Tanford C. Contribution of Hydrophobic Interactions to the Stability of the Globular Conformation of Proteins. J. Am. Chem. Soc., (1962), 84 (22), pp 4240-4247.
10. Mant C., Zhou N. E., Hodges R. S. Correlation of protein retention times in reversed-phase chromatography with polypeptide chain length and hydrophobicity. Journal of Chromatography, 476 (1989), 363-375.
11. Wilce M. J. C., Aguilar M., Heam M. T. W. Physicochemical Basis of Amino Acid Hydrophobicity Scales: Evaluation of Four New Scales of Amino Acid Hydrophobicity Coefficients Derived from RP-HPLC of Peptides. Anal. Chem. (1995), 67, 1210-1219.
12. Clare Mills E. N., Jenkins J. A., Alcocer M. J. C., Shewry P. R. Structural, Biological, and Evolutionary Relationships of Plant Food Allergens Sensitizing via the Gastrointestinal Tract. Critical Reviews in Food Science and Nutrition, 44:379-407 (2004).
13. Cui J., Han L. Y., Li H., Ung C. Y., Tang Z. Q., Zheng C. J., Cao Z. W., Chen Y. Z. Computer prediction of allergen proteins from sequence-derived protein structural and physicochemical properties. Molecular Immunology 44 (2007) 514-520.

1. A composition comprising:
(a) synthetic nanocarriers comprising:
a polyester polymer matrix and allergen,
wherein the allergen is encapsulated in the polyester polymer matrix, and
wherein the polyester polymer matrix has a calculated hydrophilic to lipophilic balance (HLB) ranging from 11 to 15;
wherein the load of the allergen is 0.5 to 2.5 wt %; and
(b) a pharmaceutically acceptable excipient.

2-15. (canceled)

16. The composition of claim 1, wherein the allergen is in the form of a purified protein.

17. The composition of claim 1, wherein the allergen is in the form of a mixture of purified proteins.

18. The composition of claim 1, wherein the allergen is in the form of an extract.

19. (canceled)

20. The composition of claim 1, wherein the weighted mean retention time (as calculated according to Equation 3) of a sample of the allergen, obtained using reverse-phase high performance liquid chromatography (RP-HPLC), is between 1 and 10 minutes.

21-30. (canceled)

31. The composition of claim 20, wherein the sample of the allergen is monitored at 200 nm absorbance.

32. The composition of claim 20, wherein the sample of the allergen injected onto a column of the instrument is a C18 UHPLC column contains 3 ug of allergen.

33. (canceled)

34. The composition of claim 20, wherein the flow rate of the RP-HPLC is 3.0 mL/minute.

35. The composition of claim 20, wherein mobile phase A of the RP-HPLC is composed of 94.9% water, 5% acetonitrile, and 0.1% trifluoroacetic acid on a volume percent basis.

36. The composition of claim 20, wherein mobile phase B of the RP-HPLC is composed of 19.9% water, 80% acetonitrile, 0.1% trifluoroacetic acid on a volume percent basis.

37. (canceled)

38. The composition of claim 20, wherein protein peaks are identified, and the area under the curve is calculated using chromatography software.

39. The composition of claim 20, wherein the weight percent of each peak is calculated based on the area under the curve and is divided by the sum of the area under the peaks for all identified protein peaks.

40-47. (canceled)

48. The composition of claim 1, wherein the polyester polymer matrix comprises poly(lactic acid) (PLA), poly(glycolic acid) (PGA), poly(lactic-co-glycolic acid) (PLGA), polyvalerolactone (PVL), or polycaprolactone (PCL).

49-51. (canceled)

52. The composition of claim 1, wherein the mean dimension, obtained using dynamic light scattering, of the synthetic nanocarriers is greater than 90 nm but less than 200 nm.

53-57. (canceled)

58. The composition of claim 1, wherein the synthetic nanocarriers are double emulsion synthetic nanocarriers.

59. A method comprising administering the composition of claim 1 to a subject in need thereof.

60. A method comprising:
producing a composition comprising synthetic nanocarriers of claim 1, and
providing the composition to a subject in need thereof.

61-63. (canceled)

64. A method comprising:
selecting a composition comprising synthetic nanocarriers as defined in claim 1, and
providing the composition to a subject in need thereof.

65-68. (canceled)

69. A composition comprising synthetic nanocarriers produced by the method of claim 60.

70. A method comprising providing the composition of claim 69 to a subject in need thereof.
